SEQUENCE LISTING (18 sequences)

SEQ ID NO: 1 - protein, 12 aa, Artificial Sequence
  Synthetic peptide
LSEGLGELMQRC

SEQ ID NO: 2 - protein, 12 aa, Artificial Sequence
  Synthetic peptide
  DISULFID (3)..(12): cyclization
VGCVFVMSQKRC

SEQ ID NO: 3 - protein, 12 aa, Artificial Sequence
  Synthetic peptide
  DISULFID (1)..(12): cyclization
CEPLLGEAWDLC

SEQ ID NO: 4 - protein, 12 aa, Artificial Sequence
  Synthetic peptide
  DISULFID (1)..(12): cyclization
CPRGLSGGWPAC

SEQ ID NO: 5 - protein, 12 aa, Artificial Sequence
  Synthetic peptide
  DISULFID (1)..(12): cyclization
CESGEGTSNMDC

SEQ ID NO: 6 - protein, 12 aa, Artificial Sequence
  Synthetic peptide
  DISULFID (3)..(12): cyclization
LLCGRMIGDMSC

SEQ ID NO: 7 - protein, 12 aa, Artificial Sequence
  Synthetic peptide
  DISULFID (1)..(12): cyclization
CDRTDAPASANC

SEQ ID NO: 8 - protein, 12 aa, Artificial Sequence
  Synthetic peptide
  DISULFID (1)..(12): cyclization
CDDDNAARDENC

SEQ ID NO: 9 - protein, 12 aa, Artificial Sequence
  Synthetic peptide
  DISULFID (1)..(12): cyclization
CQGGRLDLFGRC

SEQ ID NO: 10 - protein, 12 aa, Artificial Sequence
  Synthetic peptide
  DISULFID (2)..(12): cyclization
LCGYGETLMVSC

SEQ ID NO: 11 - protein, 17 aa, Artificial Sequence
  Synthetic peptide
  DISULFID (1)..(12): cyclization
CHPGDKQEDPNCLQADK

SEQ ID NO: 12 - protein, 12 aa, Artificial Sequence
  Synthetic peptide
  DISULFID (1)..(12): cyclization
CEGLPADWAAAC

SEQ ID NO: 13 - protein, 20 aa, Artificial Sequence
  Synthetic peptide
MRPEIWIAQELRRIGDEFNE

SEQ ID NO: 14 - protein, 20 aa, Artificial Sequence
  Synthetic peptide
MRPEIWIAQELRRIGDEFNA

SEQ ID NO: 15 - protein, 12 aa, Artificial Sequence
  Synthetic peptide
  DISULFID (1)..(12): cyclization
CSARWGPTMPWC

SEQ ID NO: 16 - protein, 12 aa, Artificial Sequence
  Synthetic peptide
  DISULFID (1)..(12): cyclization
CRRGSRASGAHC

SEQ ID NO: 17 - protein, 10 aa, Artificial Sequence
  Synthetic peptide
SARWGPIMPW

SEQ ID NO: 18 - protein, 12 aa, Artificial Sequence
  Synthetic peptide
  DISULFID (1)..(12): cyclization
CSARWGPIMPWC

(19) United States
(12) Patent Application Publication
(10) Pub. No.: US 2020/0339629 A1
(43) Pub. Date: Oct. 29, 2020

(54) THERAPEUTIC CONSTRUCTS COMPRISING CMET BINDING PEPTIDES

(71) Applicant: AEBI Ltd., Ness Ziona (IL)

(72) Inventors: Ilan MORAD, Ness Ziona (IL); Hanan ITZHAKI, Ness Ziona (IL)

(21) Appl. No.: 16/855,292

(22) Filed: Apr. 22, 2020

Related U.S. Application Data
(60) Provisional application No. 62/837,731, filed on Apr. 24, 2019.

(51) Int. Cl. C07K 7/08; A61P 35/00; A61K 47/60

(52) U.S. Cl. CPC C07K 7/08; A61K 38/00; A61K 47/60; A61P 35/00

(57) ABSTRACT

Constructs comprising a plurality of peptides are provided and their use in treating cancer. The construct optionally connected to an organic scaffold, comprise ate least one peptide capable of targeting the tumor antigen cMet, at least two peptides targeting two additional extracellular tumor antigens and a toxin. Specific peptide sequences binding to the extracellular tumor antigens cMet or EGFR are also provided.

### FIELD OF THE INVENTION

The invention relates to constructs comprising a plurality of peptides capable of targeting the extracellular tumor antigen cMET, at least two additional extracellular tumor antigens, and at least one toxin. The peptides are optionally connected to an organic scaffold. Specific peptides binding the tumor antigens cMET or EGFR are also provided as well as their conjugates with use of the toxins having antitumor activity. Use of the constructs, peptides and conjugates in treating cancer is also provided as well.

### BACKGROUND OF THE INVENTION

Targeted cancer therapies involve substances designed to interfere with specific molecules involved in cancer cell growth and survival. In contrast to traditional chemotherapy drugs, which usually act against all actively dividing cells, a primary goal of targeted therapies is to target mainly cancer cells with more precision and potentially fewer side effects. Targeted cancer therapies that have been approved for use against specific cancers include agents that prevent cell growth signaling, interfere with tumor blood vessel development, promote the death of cancer cells, stimulate the immune system to destroy cancer cells, and deliver toxic drugs to cancer cells. The latter mainly includes monoclonal antibodies that deliver toxic molecules. Once the antibody has bound to its target cell, the toxic molecule that is linked to the antibody, such as a radioactive substance, a toxic polypeptide or a poisonous chemical, is taken up by the cell, and ultimately killing that cell. The toxin does not affect cells that lack the target for the antibody.

Cells become cancer cells largely because of mutations in their genes. The mutations may affect different genes that control cell growth and division. Mutations may cause some normal genes to become cancer-causing genes known as oncogenes. It takes more than one mutation in a cell for cancer to occur and cancers characterized by several mutations are more difficult to treat and more likely to develop resistance to chemotherapy.

Efficient tumor targeting is challenging for a number of reasons. First, it requires identifying a target that is sufficiently specific to the tumor cells to avoid as much as possible non-specific killing of cells. In addition, cancer cells tend to be variable, both between cancer types and within the same type of cancer: the expression pattern of surface targets may vary between cells of a particular tumor. Cancer cells may also alter expression of their cell surface receptors during tumor development or become resistant to the therapy. Resistance may occur in two ways: the target itself, namely the tumor antigen, changes through mutation so that the targeted therapy no longer interacts well with it, and/or the tumor finds a new pathway to achieve tumor growth that does not depend on the target. Most anti-cancer drugs attack a specific target on, or in, the cancer cell. Inhibiting the target usually aims to block a physiological pathway that promotes cancer. Mutations in the targets, or in their downstream physiological pathways, make them not sufficient as targets for cell destruction. Simultaneous targeting of several tumor antigens represents a promising approach to combat multi-mutation cancer.

The receptor tyrosine kinase cMet (also known as hepatocyte growth factor receptor), and its ligand, hepatocyte growth factor (HGF), regulate multiple cellular processes that stimulate cell proliferation, invasion and angiogenesis. HGF/cMet signaling pathway was proposed as relevant target for cancer treatment based on overexpression in many types of cancer (Sierra R. and Tsao M., Ther Adv Med Oncol. 2011 November; 3(1 Suppl): S21-S35). Small molecule inhibitors of cMet (tivantinib), and c-Met/HGF antibodies (rilotumumab and MetMAb) are known.

DeNardo et al., 2003, Clin Cancer Res. 9(10 Pt 2): 3854S-64S report the synthesis of branched poly(ethylene glycol) (PEGylated) polymers (Mr 40,000, Mr 70,000, Mr 100,000, and Mr 150,000) conjugated to tumor-specific or control peptides, to assess the effect of both molecular weight and tumor specificity on pharmacokinetics and biodistribution.

Tsai et al., 2011, J Neurooncol. 103(2): 255-266, describe a bispecific ligand-directed toxin designed to simultaneously target epidermal growth factor receptor (EGFR) on human glioblastoma cells and urokinase receptor (uPAR) on tumor neovasculature. The construct is a single-chain polypeptide consisting of human epidermal growth factor (EGF), a fragment of urokinase and truncated pseudomonas exotoxin (PE38).

cGuire et al., 2014, Sci Rep., 4:4480 describe the characterization of tumor targeting peptides for non-small cell lung cancer (NSCL) identified from phage-display libraries. The peptides were synthesized as monomers and homo-tetramers.

U.S. Pat. No. 7,947,289 discloses compositions comprising modified bacterial toxins and methods for using them for targeting particular cell populations and for treating diseases.

US 2004/0058865 discloses synthetic multimeric ligands that provide enhanced cell-specific and organ-specific targeting.

US 2009/0130105 discloses compositions that bind to multiple epitopes of insulin growth factor-1 receptor (IGF-1R), for example, combinations of bispecific binding molecules. Using the binding molecules to antagonize IGF-1R signaling are also disclosed.

WO 2007/093373 discloses in vivo stable branched peptides, in particular derived from the sequence of Neurotensin (NT) and Luteinizing hormone-releasing hormone (LHRH), conjugated to functional units for specific targeting of cancer cells, either for tumor diagnosis or therapy.

WO 2018/061104 discloses multi-targeting constructs and peptides that bind epidermal growth factor receptor (EGFR) or programmed death-ligand 1 (PD-L1).

WO 2019/064297 discloses cytotoxic peptides and conjugates thereof.

There still remains an unmet need for improved compositions and methods for targeted cancer therapy, with enhanced potency and reduced adverse effects, in particular against tumors having several gene mutations.

### SUMMARY OF THE INVENTION

The present invention relates to multi-targeting constructs comprising at least one peptide that binds the receptor tyrosine kinase cMet (also known as hepatocyte growth factor receptor, HGF) and at least one toxin. The present invention also provides constructs comprising at least three different peptides binding to at least three different extracellular tumor antigens, and at least one toxin, wherein at least one of the three different peptides binds cMet, and wherein the peptides and the toxin are covalently bound directly or through a carrier. The invention is based in part on the finding that a construct comprising a peptide that binds cMet, two additional peptides binding two additional targets on cancer cells, and a toxin have improved activity in destruction of cancer cells compared to constructs that do not include cMet-binding peptides.

According to one aspect, the present invention provides a construct comprising: i) at least one peptide that binds cMet; ii) at least two different peptides binding to at least two different extracellular tumor antigens, other than cMet; and at least one toxin, wherein the peptides and the toxin are covalently bound directly or through a carrier.

According to some embodiments, at least one of the two different peptides binds specifically to an extracellular tumor antigen selected from human epidermal growth factor receptor (EGFR) and human Programmed death-ligand 1 (PD-L1). In certain embodiments, the another one of the at least two peptides binds specifically to an extracellular tumor antigen selected from the group consisting of EGFR, PD-L1, HER2, androgen receptor, benzodiazepine receptor, Cadherin, CXCR4, CTLA-4, CD2, CD19, endothelin receptor, ERBB4, FGFR, folate receptor, HER4, Mucin 1, OGFR, PD-1, PD-L2, PDGFR, and VEGFR. According to some embodiments, the construct comprises 3 to 10 different peptides binding to different extracellular tumor antigens, including the receptor cMet.

According to some specific embodiments, the construct comprises a peptide that binds cMet, a peptide that binds EGFR and a peptide that binds PD-L1.

In some embodiments, the present invention provides a construct comprising at least three different peptides binding to at least three different extracellular tumor antigens, and at least one toxin, wherein the peptides and the toxin are covalently bound directly or through a carrier and wherein at least one of the peptides binds specifically to cMet and comprises an amino acid sequence selected from the group consisting of:
LSEGLGELMQRC denoted M582 (SEQ ID NO: 1);
VGCVFVMSQKRC denoted M578, having a disulfide bridge connecting residues 3 and 12 (SEQ ID NO: 2);
CEPLLGEAWDLC denoted M571 having a disulfide bridge connecting residues 1 and 12 (SEQ ID NO: 3);

CPRGLSGGWPAC denoted M572 having a disulfide bridge connecting residues 1 and 12 (SEQ ID NO: 4); and CESGEGTSNMDC denoted M573 having a disulfide bridge connecting residues 1 and 12 (SEQ ID NO: 5). In other embodiments, the present invention provides a construct comprising a peptide that binds specifically to cMet and at least two different peptides binding to at least two different extracellular tumor antigens other than cMet, and at least one toxin, wherein the peptides and the toxin are covalently bound directly or through a carrier and wherein at least one of the at least two different peptides binds specifically to EGFR and comprises an amino acid sequence selected from the group consisting of:

LLCGRMIGDMSC denoted E9f having a disulfide bridge connecting residues 3 and 12 (SEQ ID NO: 6);
CDRTDAPASANC denoted E404 having a disulfide bridge connecting residues 1 and 12 (SEQ ID NO: 7);
CDDDNAARDENC denoted E478 having a disulfide bridge connecting residues 1 and 12 (SEQ ID NO: 8);
CQGGRLDLFGRC denoted E494 having a disulfide bridge connecting residues 1 and 12 (SEQ ID NO: 9);
LCGYGETLMVSC denoted E133f having a disulfide bridge connecting residues 2 and 12 (SEQ ID NO: 10); and CHPGDKQEDPNCLQADK denoted E13.3 (SEQ ID NO: 11).

In other embodiments, the present invention provides a construct comprising i) a peptide that binds specifically to cMet and comprises a sequence selected from the group consisting of:

LSEGLGELMQRC denoted M582 (SEQ ID NO: 1);
VGCVFVMSQKRC denoted M578, having a disulfide bridge connecting residues 3 and 12 (SEQ ID NO: 2);
CEPLLGEAWDLC denoted M571 having a disulfide bridge connecting residues 1 and 12 (SEQ ID NO: 3);
CPRGLSGGWPAC denoted M572 having a disulfide bridge connecting residues 1 and 12 (SEQ ID NO: 4); and CESGEGTSNMDC denoted M573 having a disulfide bridge connecting residues 1 and 12 (SEQ ID NO: 5);
ii) a peptide that binds specifically EGFR and comprises a sequence selected from:
CHPGDKQEDPNCLQADK denoted E13.3 (SEQ ID NO: 11);
LLCGRMIGDMSC denoted E9f having a disulfide bridge connecting residues 3 and 12 (SEQ ID NO: 6);
CDRTDAPASANC denoted E404 having a disulfide bridge connecting residues 1 and 12 (SEQ ID NO: 7);
CDDDNAARDENC denoted E478 having a disulfide bridge connecting residues 1 and 12 (SEQ ID NO: 8);
CQGGRLDLFGRC denoted E494 having a disulfide bridge connecting residues 1 and 12 (SEQ ID NO: 9); and
LCGYGETLMVSC denoted E133f having a disulfide bridge connecting residues 2 and 12 (SEQ ID NO: 10).
iii) a peptide that binds specifically PD-L1 and comprises the sequence:

### CEGLPADWAAAC (SEQ ID NO: 12).

and at least one toxin, wherein the peptides and the toxin are covalently bound directly or through a carrier or a linker.

According to any one of the above embodiments, the construct comprises multiple copies of at least one of the peptides. Any targeting peptide or peptide toxin may be present in multiple copies of the constructs of the invention. In some embodiments, the construct comprises from 2 to 50 copies of at least one of the peptides. According to any one of the above embodiments, the construct comprises multiple copies of each of the peptides. According to some embodiments, the construct comprises independently 2 to 10 copies of each of the targeting peptides. According to yet other embodiments, each of the targeting peptides is independently present in 2 to 10 copies and each of the toxins is independently present in 5-50 copies.

According to any one of the above embodiments, the toxin is a peptide, polypeptide or protein toxin. In some embodiments, the toxin is selected from a toxin binding to a eukaryotic elongation factor 2, BIM toxin selected from MRPEIWIAQELRRIGDEFNE (BIMe, SEQ ID NO: 13), and MRPEIWIAQELRRIGDEFNA (BIMa, SEQ ID NO: 14), Diphtheria toxin, Pseudomonas exotoxin, Anthrax toxin, botulinum toxin, Ricin, PAP, Saporin, Gelonin, Momordin, ProTx-I ProTx-II, Conus californicus toxin, snake-venom toxin, and cyanotoxin. In some embodiments, the toxin binding to eukaryotic elongation factor 2 is a toxin comprising the amino acid sequence selected from CSARWGPTMPWC (SEQ ID NO: 15), CRRGSRASGAHC (SEQ ID NO: 16), SARWGPIMPW (SEQ ID NO: 17), CSARWGPIMPWC (GW2, SEQ ID NO: 18) or an analog thereof. According to some embodiments, the construct comprises 2 to 10 different toxins.

According to any one of the above embodiments, the construct comprises multiple copies of at least one or of at least two of the toxins. According to one embodiment, the construct comprises from 2 to 50 copies of the at least one of the toxins. According to another embodiment, the construct comprises 2 to 50 copies of at least one peptide toxin having a sequence selected from the group consisting of: CSARWGPTMPWC (SEQ ID NO: 15), CRRGSRASGAHC (SEQ ID NO: 16), MRPEIWIAQELRRIGDEFNE (SEQ ID NO: 13), MRPEIWIAQELRRIGDEFNA (SEQ ID NO: 14), SARWGPIMPW (SEQ ID NO: 17), CSARWGPIMPWC (GW2, SEQ ID NO: 18) or an analog thereof.

According to any one of the above embodiments, at least one of the peptides and/or at least one toxin are covalently bound through a carrier. According to one embodiment, the carrier is an organic scaffold. According to another embodiment, each one of the targeting peptides and toxin(s) is bound to an organic scaffold. According to some embodiments, the scaffold is a polyethylene glycol (PEG) molecule or a modified PEG molecule. According to some embodiments, the PEG molecule is a branched molecule. According to another embodiment, the PEG molecules comprises a plurality of sites for binging the peptides and/or the toxin(s) of the present invention. According to one embodiment, the PEG molecule comprises 8 to 80 sites available to connect the peptides and the toxins. According to some specific embodiments, the PEG molecule comprises 10-60 sites connecting the peptides and toxin molecules. According to some specific embodiments, the PEG molecule comprises 56 sites connecting the peptides and toxin molecules. According to some specific embodiments, the PEG molecule comprises 8 sites to which targeting and toxin peptides are connected.

According to some embodiments, the present invention provides a construct comprising multiple copies of each one of at least three different peptides binding to at least three different extracellular tumor antigens, and at least one toxin, wherein the peptides and the toxins are bound to the scaffold, wherein at least one of the peptides binds specifically to cMet and wherein at least one of the other peptides binds an extracellular tumor antigen selected from EGFR or PD-L1. According to some embodiments, one of the peptides binds specifically to cMet, one binds specifically to EGFR and one of the peptides binds specifically to PD-L1. According to one specific embodiment, the peptide that binds specifically to EGFR comprises the sequence CHPGDKQEDPNCLQADK (SEQ ID NO: 11, denoted E13.3), the peptide that binds specifically to cMet comprises the sequence LSEGLGELMQRC (SEQ ID NO: 1), and the peptide that bind specifically PD-L1 comprises the sequence CEGLPADWAAAC (GR, SEQ ID NO: 12). According to certain embodiments, the toxin comprises an amino acid sequence selected from: CSARWGPTMPWC (SEQ ID NO: 15), CRRGSRASGAHC (SEQ ID NO: 16), MRPEIWIAQELRRIGDEFNE (BIMe, SEQ ID NO: 13), MRPEIWIAQELRRIGDEFNA (BIMa, SEQ ID NO: 14), SARWGPIMPW (SEQ ID NO: 17), CSARWGPIMPWC (GW2, SEQ ID NO: 18), or an analog thereof. According to one embodiment, the scaffold is PEG scaffold. According to one embodiment, the PEG molecule comprises 8 to 56 sites connected to targeting peptides and toxins molecules.

According to one embodiment, the construct comprises multiple copies of each one of: (i) a peptide having the sequence LSEGLGELMQRC (M582, SEQ ID NO: 1) (ii) a peptide having the sequence CHPGDKQEDPNCLQADK (E13.3, SEQ ID NO: 11), (iii) a peptide having the sequence CEGLPADWAAAC (GR, SEQ ID NO: 12), and (iv) a toxin having the sequence MRPEIWIAQELRRIGDEFNE (BIMe, SEQ ID NO: 13), wherein each one of the peptides and the toxins is bound to the scaffold or to another peptide. According to one embodiment, the scaffold is PEG scaffold. According to another embodiment, the construct comprises 7 copies of each one of the targeting peptides of (i), (ii) and (iii) and 35 copies of the toxin (iv).

According to any one of the above embodiments, at least one of the targeting peptides or of the toxins is connected to the scaffold through a linker or spacer.

According to any one of the above embodiments, the construct further comprises a permeability-enhancing moiety.

According to a further aspect, the present invention provides a peptide comprising an amino acids sequence selected from the group consisting of: LSEGLGELMQRC denoted M582 (SEQ ID NO: 1); VGCVFVMSQKRC denoted M578, having a disulfide bridge connecting residues 3 and 12 (SEQ ID NO: 2); CEPLLGEAWDLC denoted M571 having a disulfide bridge connecting residues 1 and 12 (SEQ ID NO: 3); CPRGLSGGWPAC denoted M572 having a disulfide bridge connecting residues 1 and 12 (SEQ ID NO: 4); CESGEGTSNMDC denoted M573 having a disulfide bridge connecting residues 1 and 12 (SEQ ID NO: 5); or an analog or salt thereof. According to one embodiment, the peptide or the analog binds the human protein cMet. According to another embodiment, the peptide or the analog is cyclic. According to one embodiment, the peptide or the analog is for use in targeting cancer cells.

According to another aspect, the present invention provides a peptide comprising an amino acids sequence selected from the group consisting of: LLCGRMIGDMSC denoted E9f having a disulfide bridge connecting residues 3 and 12 (SEQ ID NO: 6); CDRTDAPASANC denoted E404 having a disulfide bridge connecting residues 1 and 12 (SEQ ID NO: 7); CDDDNAARDENC denoted E478 having a disulfide bridge connecting residues 1 and 12 (SEQ ID NO: 8); CQGGRLDLFGRC denoted E494 having a disulfide bridge connecting residues 1 and 12 (SEQ ID NO: 9); LCGYGETLMVSC denoted E133f having a disulfide bridge connecting residues 2 and 12 (SEQ ID NO: 10); or an analog or salt thereof. According to one embodiment, the peptide or the analog binds the human protein Epidermal Growth Factor Receptor (EGFR). According to another embodiment, the peptide or the analog is cyclic. According to one embodiment, the peptide or the analog is for use in targeting cancer cells.

According to yet other embodiments, the invention provides a toxin peptide comprising the sequence MRPEIWIAQELRRIGDEFNE (BIMe, SEQ ID NO: 13), or an analog or a salt thereof.

According to certain aspects, the present invention provides a conjugate comprising at least one peptide described above that binds cMet or EGFR. According to some embodiments, the conjugate comprises a peptide that binds cMet, having a sequence selected from the group consisting of: SEQ ID NO: 1, SEQ ID NO: 2 SEQ ID NO: 3, SEQ ID NO: 4, SEQ ID NO: 5, or an analog or salt thereof. According to other embodiments, the conjugate comprises a peptide that binds EGFR, having a sequence selected from the group consisting of: SEQ ID NO: 6, SEQ ID NO: 7 SEQ ID NO: 8, SEQ ID NO: 9, SEQ ID NO: 10, or an analog or salt thereof.

The novel peptides and conjugates of the present invention, which bind to the tumor antigens cMet or EGFR may be used for detection of these proteins. In particular, the conjugates of the cMet-binding or EGFR-binding peptides may be used to identify and quantify tumor cells and metastases and for diagnosis of cancer.

According to another aspect, the present invention provides a composition comprising a peptide, a peptide conjugate or a construct of the present invention. According to one embodiment, the composition is a pharmaceutical composition. Thus, in some embodiments, the present invention provides a pharmaceutical composition comprising a peptide or a construct described above and a pharmaceutically acceptable excipient. According to one embodiment, the pharmaceutical composition comprises a construct comprising multiple copies each of: a peptide that binds cMet, at least two additional peptides binding to two different extracellular tumor antigens, and at least one toxin, wherein the peptides and the toxins are bound to the scaffold directly or through a linker or a spacer and wherein at least one of the two additional peptides binds specifically to the extracellular tumor antigens selected from EGFR or PD-L1. According to some embodiments, one of the peptides binds specifically to EGFR and one of the peptides binds specifically to PD-L1. According to some embodiments, the scaffold is PEG scaffold comprising 6 to 80 arms connected with peptides.

According to other embodiments, the pharmaceutical composition comprises a peptide that binds cMet having a sequence selected from the group consisting of: SEQ ID NO: 1, SEQ ID NO: 2 SEQ ID NO: 3, SEQ ID NO: 4, SEQ ID NO: 5, or an analog or salt thereof.

According to other embodiments, the pharmaceutical composition comprises a peptide that binds EGFR having a sequence selected from the group consisting of: SEQ ID NO: 6, SEQ ID NO: 7 SEQ ID NO: 8, SEQ ID NO: 9, SEQ ID NO: 10, or an analog or salt thereof.

According to other embodiments, the pharmaceutical composition comprises a peptide toxin comprising the sequence MRPEIWIAQELRRIGDEFNE (SEQ ID NO: 13) or an analog or salt thereof.

According to yet other embodiments, the pharmaceutical composition comprises a conjugate of a peptide that binds cMet, of a peptide that binds EGFR, or of a peptide toxin described above.

According to one embodiment, the pharmaceutical composition comprises a construct comprising multiple copies of each one of the: (i) a peptide having the sequence LSEGLGELMQRC (SEQ ID NO: 1), (ii) a peptide having the sequence CEGLPADWAAAC (SEQ ID NO: 12), (iii) a peptide having the sequence CHPGDKQEDPNCLQADK (SEQ ID NO: 11), and (iv) a toxin having the sequence MRPEIWIAQELRRIGDEFNE (SEQ ID NO: 13), wherein each one of the peptides and the toxins is bound to the scaffold. According to one embodiment, the scaffold is PEG scaffold.

According to some embodiments, the pharmaceutical composition of the present invention is for use in treating cancer.

According to other embodiments, the pharmaceutical composition of the present invention is for use in diagnosing cancer or identifying cancer cells.

The present invention also provides a method of detecting cancer cells that over express at least one of the extracellular proteins cMet and EGFR, the method comprising contacting a sample with at least one peptide described above that binds cMet or EGFR, or with a construct described above comprising at least one peptide that binds cMet or EGFR. According to some embodiments, the construct comprises multiple copies of at least one peptide that binds cMet and multiple copies of at least one peptide that binds EGFR. According to other embodiments, the construct further comprises multiple copies of at least one peptide that binds PD-L1.

According to certain aspects, the present invention provides a method of treating cancer in a subject in need thereof comprising administering to said subject a pharmaceutical composition comprising a construct or a peptide described above. According to some embodiments, the present invention provides a method of treating cancer in a subject in need thereof comprising administering a therapeutically effective amount of a peptide that binds cMet or EGFR or a construct comprising a peptide that binds cMet and/or EGFR.

According to some embodiments, a cancer amendable with the peptides and constructs of the present invention is characterized by over-expressing the tumor antigen cMet and at least one additional tumor antigen. According to some embodiments, the at least one additional tumor antigen is selected from the group consisting of: EGFR, PD-L1, HER2, androgen receptor, benzodiazepine receptor, Cadherin, CXCR4, CTLA-4, CD2, CD19, endothelin receptor, ERBB4, FGFR, folate receptor, HER4, Mucin 1, OGFR, PD-1, PD-L2, PDGFR, and VEGFR.

According to some embodiments, the cancer cells treatable with the constructs of the present invention over-express the tumor antigens cMet, EGFR and PD-L1.

According to some embodiments, the present invention provides a method of treating cancer selected from the group consisting of: lung cancer, colon cancer, CNS cancer, pancreatic cancer, large intestine/bowel cancer, head and neck cancer, esophagus cancer, ovarian cancer, bone cancer, kidney cancer, stomach cancer, comprising administering to a subject in need thereof, a composition comprising at least one construct according to the present invention.

According to a further aspect, the present invention provides an isolated polynucleotide comprising a sequence encoding the peptide of the present invention. According to one embodiment, the polynucleotide encodes a peptide that binds cMet having a sequence selected from the group consisting of: VGCVFVMSQKRC (M578, SEQ ID NO: 2), LSEGLGELMQRC (M582, SEQ ID NO: 1), CEPLLGEAWDLC (M571, SEQ ID NO: 3), CPRGLSGGWPAC (M572, SEQ ID NO: 4) and CESGEGTSNMDC (M573, SEQ ID NO: 5).

According to one embodiment, the polynucleotide encodes a peptide that binds EGFR having a sequence selected from the group consisting of: LLCGRMIGDMSC (E9f, SEQ ID NO: 6), CDRTDAPASANC (E404, SEQ ID NO: 7), CDDDNAARDENC (E478, SEQ ID NO: 8), CQGGRLDLFGRC (E494, SEQ ID NO: 9), and LCGYGETLMVSC (E133f, SEQ ID NO: 10).

According to yet other embodiments, the polynucleotide encodes a peptide toxin comprising the sequence MRPEIWIAQELRRIGDEFNE (BIMe, SEQ ID NO: 13).

According to yet another aspect, the present invention provides a nucleic acid construct comprising the polynucleotide of the present invention. According to one embodiment, the polynucleotide is operably linked to a promoter.

According to certain aspects, the present invention provides a vector comprising at least one polynucleotide or at least one nucleic acid construct of the present invention.

According to a further aspect, the present invention provides a cell comprising at least one polynucleotide or at least one nucleic acid construct of the present invention.

### BRIEF DESCRIPTION OF THE FIGURES

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

FIG. 1A shows microscopic images of A549 cells (i) before incubation with test items; (ii) after 48 hours incubation with the construct PEG-E404-GW2 3 μM (positive control); and (iii) after 48 hours incubation with PBS (negative control).

FIGS. 1B-1E show microscopic images of A549 cells following 48 hours incubation with the constructs: PEG-M582-BIMe (FIG. 1B); PEG-M571-BIMe (FIG. 1C); PEG-M572-BIMe (FIG. 1D); and PEG-M573-BIMe (FIG. 1E), each one in concentrations of: (i) 9 μM; (ii) 3 μM; and (iii) 1 μM.

FIG. 2A shows microscopic images of A549 cells (i) before incubation with test items; (ii) after 48 hours incubation with the construct PEG-E404-GW2 3 μM (positive control); and (iii) after 48 hours incubation with PBS (negative control).

FIGS. 2B-2F show microscopic images of A549 cells following 48 hours incubation with the constructs: PEG-E9f-BIMe (FIG. 2B), PEG-E404-BIMe (FIG. 2C), PEG-E478-BIMe (FIG. 2D), PEG-E494-BIMe (FIG. 2E) and PEG-E133f-BIMe (FIG. 2F), each one in concentrations of: (i) 9 μM; (ii) 3 μM; and (iii) 1 μM.

FIG. 3 shows microscopic images of A549 cells following 48 hours incubation with the construct PEG-E13.3-GR-M582-BIMe in concentrations of: (i) 3 μM; (ii) 1 μM; (iii) 0.3 μM; and (iv) 0.1 μM.

FIG. 4 is a graph depicting the mean tumor size vs. time (days) of tumor xenograft induction in mice. Subcutaneous tumors containing A549 lung cancer cells (overexpressing EGFR, PDL1 and cMet) were induced in 12 mice, 6 mice were injected IV with a construct (filled circles), that contained targeting peptides to EGFR, PDL1, cMet, and a toxic peptide (E13.3, GR, M582, BIM respectively), 6 mice served as a control group (triangles) and were injected with PBS. Each point of the graph represents the mean tumor volume of 5 mice, 4 of them are statistically different by T test (P<0.05).

### DETAILED DESCRIPTION OF THE INVENTION

The present invention relates to therapeutic constructs comprising plurality of multi-target peptides and at least one toxin moiety. In particular, a construct according to the present invention comprises a plurality of peptides each directed against a different cell-target. Peptides contained in a construct according to the invention are capable of binding, blocking, inhibiting, and/or activating at least three different antigens, including the antigen cMet, expressed on the membrane of cancer cells. The present invention provides, according to one aspect, a construct comprising a peptide that binds cMet and at least two additional peptides binding to at least two different extracellular tumor antigens other than cMet, and at least one toxin, wherein the peptides and the toxin are covalently bound directly or through a carrier. The present invention also provides peptides that bind cMet and peptides that bind EGFR and their uses for treatment and diagnosis of cancer and as part of the constructs and conjugates.

The term “peptide” refers to a short chain of amino acid residues linked by peptide bonds, i.e., a covalent bond formed between the carboxyl group of one amino acid and an amino group of an adjacent amino acid. The term “peptide” refers to short sequences having at least 3 and up to 50 amino acids. A chain of amino acids monomers longer than 50 amino acid is referred as a “polypeptide”. Such polypeptides, when having more than 50 amino acid residues and a tertiary structure, are also classified as proteins.

The term “peptide” encompasses also the term “peptide analog”. The term “peptide analog” and “analog” are used herein interchangeably and refer to an analog of a peptide having at least 70% identity with the original peptide, wherein the analog retains the activity of the original peptide. Thus, the terms “analog” and “active analog” may be used interchangeably. The term “analog” refers to a peptide which contains substitutions, rearrangements, deletions, additions and/or chemical modifications in the amino acid sequence of the parent peptide. According to some embodiments, the peptide analog has at least 80%, at least 90% or at least 95% sequence identity to the original peptide. According to one embodiment, the analog has about 70% to about 95%, about 80% to about 90% or about 85% to about 95% sequence identity to the original peptide. According to some embodiments, the analog of the present invention comprises the sequence of the original peptide in which 1, 2, or 3 deletions, additions and/or substitutions were made.

The term “peptide” encompasses also the term “peptide fragment”. The term “fragment” refers to a fragment of the original peptide or of an analog thereof in which 1 to 3 amino acid residues have been deleted, wherein said fragment retains the activity of the original peptide or analog. Thus, the terms “fragment” and “active fragment” may be used interchangeably.

The substitutions of the amino acids may be conservative or non-conservative substitution. The non-conservative substitution encompasses substitution of one amino acid by any other amino acid. In one particular embodiment, the amino acid is substituted by a non-natural amino acid.

The term “analog” encompasses also the term “conservative analog”. Conservative substitutions of amino acids as known to those skilled in the art are within the scope of the present invention. Conservative amino acid substitutions include replacement of one amino acid with another having the same type of functional group or side chain, e.g., aliphatic, aromatic, positively charged, negatively charged. One of skill will recognize that individual substitutions, is a “conservatively modified analog” where the alteration results in the substitution of an amino acid with a chemically similar amino acid. Conservative substitution tables providing functionally similar amino acids are well known in the art. One typical example of conservative substitution is provided below.

The following six groups each contain amino acids that are conservative substitutions for one another: (1) Alanine (A), Serine (S), Threonine (T); (2) Aspartic acid (D), Glutamic acid (E); (3) Asparagine (N), Glutamine (Q); (4) Arginine (R), Lysine (K); (5) Isoleucine (I), Leucine (L), Methionine (M), Valine (V); and (6) Phenylalanine (F), Tyrosine (Y), Tryptophan (W). In other embodiments, the conservative substitution encompass substitution with a chemically similar non-natural amino acid.

Thus, in some embodiments, the analog is a conservative analog of the peptide. According to some embodiments, the conservative analog of the present invention comprises the sequence of the original peptide in which 1, 2 or 3 conservative substitutions were made. According to another embodiment, the analog consists of the amino acid sequence of the original peptide in which 1, 2 or 3 conservative substitution were made. Thus, the analog consists of the amino acid sequence of the original peptide with 1, 2 or 3 conservative substitutions.

Also included within the scope of the invention are salts and derivatives of the peptides. As used herein the term “salts” refers to both salts of carboxyl groups and to acid addition salts of amino or guanidino groups of the peptide molecule. Salts of carboxyl groups may be formed by means known in the art and include inorganic salts, for example sodium, calcium, ammonium, ferric or zinc salts, and the like, and salts with organic bases such as salts formed for example with amines such as triethanolamine, piperidine, procaine, and the like. Acid addition salts include, for example, salts with mineral acids such as, for example, acetic acid or oxalic acid. Salts describe here also ionic components added to the peptide solution to enhance hydrogel formation and/or mineralization of calcium minerals.

“Derivatives” of the peptides of the invention as used herein covers derivatives which may be prepared from the functional groups which occur as side chains on the residues or the

N- or C-terminal groups, by means known in the art, and are included in the invention as long as they remain pharmaceutically acceptable, i.e., they do not destroy the activity of the peptide, do not confer toxic properties on compositions containing it, and do not adversely affect the immunogenic properties thereof.

These derivatives may include, for example, aliphatic esters of the carboxyl groups, amides of the carboxyl groups produced by reaction with ammonia or with primary or secondary amines, N-acyl derivatives of free amino groups of the amino acid residues formed by reaction with acyl moieties (e.g., alkanoyl or aroyl groups), or O-acyl derivatives of free hydroxyl group (e.g., that of seryl or threonyl residues) formed by reaction with acyl moieties. The term “amino acid” as used herein refers to an organic compound comprising both amine and carboxylic acid functional groups, which may be either a natural or non-natural amino acid. The twenty two natural amino acids are aspartic acid (Asp), tyrosine (Tyr), leucine (Leu), tryptophan (Trp), arginine (Arg), valine (Val), glutamic acid (Glu), methionine (Met), phenylalanine (Phe), serine (Ser), alanine (Ala), glutamine (Gln), glycine (Gly), proline (Pro), threonine (Thr), asparagine (Asn), lysine (Lys), histidine (His), isoleucine (Ile), cysteine (Cys), selenocysteine (Sec), and pyrrolysine (Pyl). Non-limiting examples of non-natural amino acids include diaminopropionic acid (Dap), diaminobutyric acid (Dab), ornithine (Orn), aminoadipic acid, β-alanine, 1-naphthylalanine, 3-(1-naphthyl)alanine, 3-(2-naphthyl)alanine, γ-aminobutiric acid (GABA), 3-(aminomethyl) benzoic acid, p-ethynyl-phenylalanine, p-propargly-oxy-phenylalanine, m-ethynyl-phenylalanine, p-bromophenylalanine, p-iodophenylalanine, p-azidophenylalanine, p-acetylphenylalanine, azidonorleucine, 6-ethynyl-tryptophan, 5-ethynyl-tryptophan, 3-(6-chloroindolyl)alanine, 3-(6-bromoindolyl)alanine, 3-(5-bromoindolyl)alanine, azidohomoalanine, p-chlorophenylalanine, α-aminocaprylic acid, O-methyl-L-tyrosine, N-acetylgalactosamine-α-threonine, and N-acetylgalactosamine-α-serine. According to one embodiment, the substitution is substitution with a non-natural amino acid. The peptides, analogs and salts of present invention may be produced by any method known in the art, including recombinant (for peptides consisting of genetically encoded amino acids) and synthetic methods. Synthetic methods include exclusive solid phase synthesis, partial solid phase synthesis, fragment condensation, or classical solution synthesis. Solid phase peptide synthesis procedures are well known to one skilled in the art. Synthetic methods to produce peptides include but are not limited to FMOC solid phase peptide synthesis described, for example in Fields G. B., Noble R., Int. J. Pept. Protein Res., 35: 161-214, 1990. Methods for synthesizing peptides on PEG are described for example in DeNardo et al. Ibid.

In some embodiments, synthetic peptides are purified by preparative high performance liquid chromatography and the peptide sequence is confirmed via amino acid sequencing by methods known to one skilled in the art.

In some embodiments, recombinant protein techniques, well known in the art, are used to generate peptides and peptide multimers (consisting of genetically encoded amino acids) of the present invention.

As used herein, the term “toxin” refers to a peptide or polypeptide substance which is poisonous, harmful or toxic (cytotoxic) to cells, such as mammalian or particularly human cells. The toxin according to the present invention may be originated from living organism such as a microorganism, plant, or higher organism, or which may be synthetically prepared, produced, or designed using any known technique, for example as described in WO 2007/010525. The toxin typically interacts with cellular biological macromolecules such as enzymes and receptors to mediate its effect. The term encompasses biologically active subunits or fragments of a toxin. According to certain embodiments, the toxin is a peptide toxin, consisting of up to 50 amino acids. According to some embodiments, the toxin being a peptide may be a cyclic peptide. Within a construct of the present invention, a toxin moiety confers at least some of its properties to the construct, and the construct mediates poisonous or harmful effects on the target cells. None limiting examples of peptide toxin include cyanobacteria toxins such as Microcystins and Nodularins, ProTx-I and ProTx-II toxins, snake venom-derived disintegrins such as Viperistatin or fragments thereof, and Hm-1 and Hm-2 toxins.

The term “carrier” refers to any molecule that covalently binds or capable of binding to the at least two different peptides and/or a toxin. Several possible binding arrangements are encompassed. According to one embodiment, one peptide and one toxin are bound via a carrier and the second peptide is bound directly to the first peptide or to the toxin. According to another embodiment, two peptides are bound via a carrier, and the toxin is bound to one of the peptides. According to a further embodiment, all peptides and toxin(s) are covalently connected to a carrier.

According to any one of the above embodiments, the peptides and/or the toxin(s) are connected via a linker. The terms “linker” and “spacer” are used herein interchangeably and refer to any molecule that covalently binds and therefore linking two molecules. Non-limiting examples of the linker are amino acids, peptides, or any other organic substance that can be used to allow distance between two linked molecules.

As used herein, the terms “target” and “cell target” refer to molecules found on cancer cells that may be a marker of cancer cell and may be involved in cancer cell growth, proliferation, survival and metastasis development. Particular examples of targets include cell-surface proteins, which upon binding to their counterparts, such as ligands, initiate a cascade that promotes tumor growth and development. A target according to the present invention is optionally highly expressed on cancer cells and not found, or found in substantially lower levels, on normal non-cancerous cells. The term “target” encompasses therefore the term “extracellular tumor antigen”. The term “tumor antigen” or “extracellular tumor antigen” are used herein interchangeably and include both tumor associated antigens (TAAs) and tumor specific antigens (TSAs). A tumor-associated antigen means an antigen that is expressed on the surface of a tumor cell in higher amounts than is observed on normal cells or an antigen that is expressed on normal cells during fetal development. A tumor specific antigen is an antigen that is unique to tumor cells and is not expressed on normal cells. The term tumor antigen includes TAAs or TSAs that have been already identified and those that have yet to be identified and includes fragments, epitopes and any and all modifications to the tumor antigens.

As used herein, the term “cell-targeting”, when referring to a moiety, particularly a peptide, that is part of a construct of the present invention, indicates that the peptide provides cell-, tissue- or organ-specific targeting. In particular, a cell-targeting peptide specifically recognizes and binds a cell target on cancer cells. By virtue of its binding, the cell-targeting peptide directs the entire construct to the cancerous tissue, to facilitate specific killing/inhibition of cancerous cells. Killing/inhibition of cancerous cells is typically affected via the toxin present in the construct, but in some embodiments, it may be affected directly by the binding of the cell-targeting peptide.

In one embodiment, the present invention provides a construct comprising a peptide that binds cMet and at least two different peptides binding to at least two different extracellular tumor antigens other than cMet, and at least one toxin, wherein the peptides and the toxin are covalently connected directly or through a carrier. The construct thus comprises three or more different peptides binding to three or more different extracellular tumor antigens including cMet. According to another embodiment, the construct comprises 4 or more different peptides binding to 4 or more different extracellular tumor antigens.

Not limiting examples of the extracellular tumor antigens are EGFR, PD-L1, HER2, androgen receptor, benzodiazepine receptor, Cadherin, CXCR4, CTLA-4, CD2, CD19, endothelin receptor, ERBB4, FGFR, folate receptor, HER4, Mucin 1, OGFR, PD-1, PD-L2, PDGFR, and VEGFR, thus according to one embodiment, at least one of the peptides binds specifically to one such extracellular tumor antigen.

According to some embodiments, at least one of the peptides binds specifically to an extracellular tumor antigens selected from Epidermal Growth Factor Receptor (EGFR) and programmed death-ligand 1 (PD-L1). The terms “PD-L1” and “human PD-L1” are used herein interchangeably. The terms “EGFR” and “human EGFR” are used herein interchangeably.

According to other embodiments, the other one of the peptides binds specifically to an extracellular tumor antigen selected from the group consisting of EGFR, PD-L1, HER2, androgen receptor, benzodiazepine receptor, Cadherin, CXCR4, CTLA-4, CD2, CD19, endothelin receptor, ERBB4, FGFR, folate receptor, HER4, Mucin 1, OGFR, PD-1, PD-L2, PDGFR, and VEGFR.

According to a further embodiment, at least one of the peptides binds specifically to EGFR and at least one peptide binds specifically to PD-L1.

According to any one of the above embodiments, the peptide consists of 5 to 30 amino acids. According to other embodiments, each peptide consists of 6 to 25 amino acids. According to yet other embodiments, each peptide consists of 7 to 20 amino acids. According to some embodiments, the peptide consists of 8 to 30 amino acids. According to some embodiments, each peptide consists of 8, 9, 10, 11, 12, 13, 14, 15, 16, 17, 18, 19 or 20 amino acids. Each possibility represents a separate embodiment of the invention. According to some embodiments, the construct comprises peptides having different lengths.

According to any one of the above embodiments, the targeting or toxic peptide of the present invention is a cyclic peptide. The terms “cyclic peptide” and “cyclopeptide” are used herein interchangeably and refer to a peptide having an intramolecular bond between two non-adjacent amino acids. The cyclization can be made through a covalent or non-covalent bond. Intramolecular bonds include, but are not limited to, backbone to backbone, side-chain to backbone and side-chain to side-chain bonds. According to some embodiments, the cyclization occurs between the cysteines of the peptide sequence. According to other embodiments, the cyclization occurs between the N-terminal and C-terminal amino acids.

According to any one of the above embodiments, the construct comprises three or more peptides binding to three or more different extracellular tumor antigens, wherein at least one of the tumor antigens is cMet. According to some embodiments, the construct comprises 3 to 10 different peptides binding to 3 to 10 different extracellular tumor antigens. According to other embodiments, the construct comprises 3 to 8, 3 to 10, or 4 to 6 different peptides. According to a further embodiment, the construct comprises 3 different peptides binding to 3 different extracellular tumor antigens. According to another embodiment, the construct comprises 4 different peptides binding to 4 different extracellular tumor antigens. According to certain embodiments, the construct comprises 5, 6, 7 or 8 different peptides binding to 5, 6, 7 or 8 different extracellular tumor antigens, respectively.

According to any one of the above embodiments, the construct of the present invention comprises multiple copies of at least one of the different peptides.

According to other embodiments, the construct of the present invention comprises multiple copies of each one of the at least two of the different peptides. According to another embodiment, the construct comprises multiple copies of each one of the peptides.

The term “different peptides” refer to peptides binding to different binding site and not to two copies of the same peptide.

According to some embodiments, the construct comprises from 2 to 100, 3 to 90, 4 to 60, 5 to 50, 6 to 40, 7 to 35, 8 to 30, 9 to 25 or 10 to 20 copies of a peptide. According to one embodiment, the construct comprises 2 to 50 copies of a peptide. According to another embodiment, the construct comprises from 7 to 56, from 14 to 48, from 21 to 42 from 28 to 35, from 7 to 21 copies of a peptide. According to other embodiments, the construct comprises 2 to 100, 3 to 90, 4 to 60, 5 to 50, 6 to 40, 7 to 35, 8 to 30, 9 to 25 or 10 to 20 copies of each one of the two different peptides. According to one embodiment, the construct comprises 2 to 50 copies of each one of the two different peptides. According to some embodiments, the construct comprises from 7 to 56, from 14 to 48, from 21 to 42 from 28 to 35, from 7 to 21 copies of each one of the two different peptides. In certain embodiments, the contract comprises from 7 or from 14 to 28 copies of each one of the 3, 4 or 5 different peptides.

According to some embodiments, the construct comprises multiple copies of a peptide that binds specifically to cMet and/or multiple copies of a peptide that binds specifically to EGFR and/or to PD-L1.

According to some embodiments, the construct comprises from 2 to 100, 3 to 90, 4 to 60, 5 to 50, 6 to 40, 7 to 35, 8 to 30, 9 to 25 or 10 to 20 copies of at least one peptide selected from the group consisting of: M578 (SEQ ID NO: 2), M582 (SEQ ID NO: 1), M571 (SEQ ID NO: 3), M572 (SEQ ID NO: 4), M573 (SEQ ID NO: 5), E9f (SEQ ID NO: 6), E404 (SEQ ID NO: 7), E478 (SEQ ID NO: 8), E494 (SEQ ID NO: 9), E133f (SEQ ID NO: 10), E13.3 (SEQ ID NO: 11) and GR (SEQ ID NO: 12), or of an analog thereof.

According to any one of the above embodiments, the toxin is selected from a peptide toxin, polypeptide toxin or peptide toxin.

According to some embodiments, the toxin is selected from the group consisting of a toxin binding to a eukaryotic elongation factor 2 or analog of that toxins, BIM-BH3 toxin, BIMe (SEQ ID NO: 13), BIMa (SEQ ID NO: 14), Diphtheria toxin, Pseudomonas exotoxin, Anthrax toxin, botulinum toxin, Ricin, PAP, Saporin, Gelonin, Momordin, ProTx-I ProTx-II, Conus californicus toxin, snake-venom toxin, and cyanotoxin. According to some embodiments, the BIM toxin consists of the amino acid sequence MRPEIWIAQELRRIGDEFNE (BIMe, SEQ ID NO: 13).

According to some embodiments, the toxin binding to eukaryotic elongation factor 2 is a toxin having the amino acid sequence selected from the group consisting of: CSARWGPTMPWC or CRRGSRASGAHC, or an analog thereof.

According to another embodiment, the toxin is selected from the group consisting a toxin having the sequence CSARWGPTMPWC (SEQ ID NO: 15), a toxin having the sequence CRRGSRASGAHC (SEQ ID NO: 16), a toxin having the sequence MRPEIWIAQELRRIGDEFNE (SEQ ID NO: 13), a toxin having the sequence MRPEIWIAQELRRIGDEFNA (SEQ ID NO: 14), a toxin having the sequence SARWGPIMPW (SEQ ID NO: 17), a toxin having the sequence CSARWGPIMPWC (SEQ ID NO: 18), Diphtheria toxin, Pseudomonas exotoxin, Anthrax toxin, botulinum toxin, Ricin, PAP, Saporin, Gelonin, Momordin, ProTx-I ProTx-II, Conus californicus toxin, snake-venom toxin, and cyanotoxin.

According to some such embodiments, the toxin or analog thereof is cyclic toxin or analog.

According to some embodiments, the construct comprises 2 to 10 different toxins. According to one embodiment, the construct comprises 2 different toxins. According to another embodiment, the construct comprises 3 different toxins. According to a further embodiment, the construct comprises 4, 5, 6, 7, 8, 9 or 10 different toxins.

According some embodiments, the construct of the present invention comprises multiple copies of at least one of the toxins. According to other embodiment, the construct comprises multiple copies of at least two toxins.

According to some embodiments, the construct comprises from 2 to 100, 3 to 90, 4 to 60, 5 to 50, 6 to 40, 7 to 35, 8 to 30, 9 to 25 or 10 to 20 copies of a toxin. According to one embodiment, the construct comprises 2 to 50 copies of a toxin. According to another embodiment, the construct comprises from 7 to 56, from 14 to 48, from 21 to 42 from 28 to 35, from 7 to 21 copies of a toxin. According to other embodiments, the construct comprises 2 to 100, 3 to 90, 4 to 60, 5 to 50, 6 to 40, 7 to 35, 8 to 30, 9 to 25 or 10 to 20 copies of each one of two different toxins. According to one embodiment, the construct comprises 2 to 50 copies of each one of two different toxins. According to some embodiments, the construct comprises from 7 to 56, from 14 to 48, from 21 to 42 from 28 to 35, from 7 to 21 copies of each one of two different toxins. In certain embodiments, the contract comprises from 7 or from 14 to 28 copies of each one of the 3, 4 or 5 different toxins.

According to some embodiments, the present invention provides a construct described above comprising a peptide that binds cMet and at least two different peptides binding to at least two different extracellular tumor antigens, and at least one toxin, wherein one of the peptides binds specifically to EGFR and one of the peptides binds specifically to PD-L1 and the toxin is selected from a toxin binding to a eukaryotic elongation factor 2, BIM toxin having an amino acid sequence selected from MRPEIWIAQELRRIGDEFNE (BIMe, SEQ ID NO: 13) and MRPEIWIAQELRRIGDEFNA (BIMa, SEQ ID NO: 14), Diphtheria toxin, Pseudomonas exotoxin, Anthrax toxin, botulinum toxin, Ricin, PAP, Saporin, Gelonin, Momordin, ProTx-I ProTx-II, Conus californicus toxin, snake-venom toxin, cyanotoxin, and any combination thereof. According to some embodiments, the toxin is a toxin binding to eukaryotic elongation factor 2.

According to some embodiments, the different targeting peptides of a construct of the present invention have a synergistic cytotoxicity. According to some such embodiments, at least one of the targeting peptides and/or the toxins, are cyclic.

According to any one of the above embodiments, the peptides of the present invention are covalently connected to each other. According to one embodiment, the peptides and the toxins are connected directly, i.e. without a carrier. According to other embodiments, the peptides of the present invention are covalently connected through a carrier or a linker. According to one embodiment, the carrier is an organic scaffold, thus the peptides are covalently connected through a scaffold.

According to some embodiments, the scaffold is a peptidic or polypeptidic scaffold. According to other embodiments, the peptidic or polypeptidic scaffold connects the peptides to each other on a single location in the scaffold, or to a different location on a scaffold. Each possibility represents a separate embodiment of the invention. According to some embodiments, the scaffold comprises at least one Lysine (Lys) residue. According to other embodiments, the scaffold comprises at least three Lys residues. According to further embodiments, the at least three Lys residues are connected together by amide bonds to form a branched multimeric scaffold. According to some embodiments, at least one amide bond is formed between the epsilon amine of a Lys residue and the carboxy group of another Lys residue.

According to a particular embodiment, the construct comprises a molecule according to one of the schemes presented below,

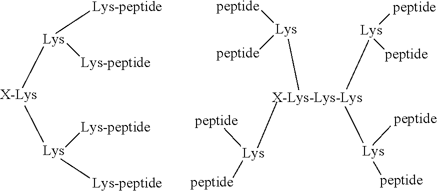

wherein X represents the peptide's and/or the toxin's C-terminal selected from carboxy acid, amide or alcohol group and optionally a linker or spacer, and peptide denotes a peptide according to the present invention, e.g. having 7-20 amino acids capable of binding to a cell-target. Each possibility represents a separate embodiment of the present invention.

According to some specific embodiments, at least one of the peptides and/or the toxin(s) is present in multiple copies. According to some embodiments, the multiple copies are linked thereby forming a multi-target peptide multimer. According to some embodiments, the peptide and/or the toxin(s) copies are linked through a linker. According to other embodiments, the peptides and/or the toxin(s) copies are linked directly. According to further embodiments, the multimer comprises copies linked both directly and via a linker.

According to some embodiments, the construct comprises a peptide multimer comprising a plurality of cell-targeting peptides arranged in an alternating sequential polymeric structure B(X₁X₂X₃ . . . Xₘ)ₙB or in a block copolymer structure B(X₁)_{nZ}(X₂)_{nZ}(X₃)ₙZ . . . (Xₘ)ₙ, wherein B is an optional sequence of 1-10 amino acid residues; n is at each occurrence independently an integer of 2-50; m is an integer of 3-50; each of X₁, X₂ . . . Xₘ is an identical or different peptide consisting of 5-30 amino acid residues; Z at each occurrence is a bond or a spacer of 1-4 amino acid residues. According to particular embodiments, n is at each occurrence independently an integer of 2-10; m is an integer of 3-10; each of X₁, X₂ . . . Xₘ is an identical or different peptide consisting of 7-20 amino acid residues; Z at each occurrence is a bond or a spacer of 1-4 amino acid residues. Each possibility represents a separate embodiment of the present invention.

According to some embodiments, the peptide multimer comprises 2-8 different peptides. According to a particular embodiment, the peptide multimer comprises 4-10 copies of a single peptide sequence. According to yet other embodiments, the peptide multimer consists of 2-10, 3-9, 4-8, or 10-100 different or identical peptides. Each possibility represents a separate embodiment of the present invention.

According to other embodiments, the scaffold comprises or formed from a polyethylene glycol (PEG) molecule(s) or a modified PEG molecule(s). According to certain embodiments, the scaffold comprises a branched PEG molecule. According to some embodiments, the branched molecule comprises at least two sites available to bind a peptide of the present invention. According to other embodiments, the scaffold comprises from 2 to 100, 3 to 90, 4 to 60, 5 to 50, 6 to 40, 7 to 35, 8 to 30, 9 to 25 or 10 to 20, or 2 to 50 sites available to bind a peptide. According to one embodiment, the construct comprises from 7 to 56, from 14 to 48, from 21 to 42 from 28 to 35, from 7 to 21 sites available to bind a peptide. According to certain embodiment, the scaffold comprises 8 or 56 sites available to bind a peptide. According to further embodiments, the scaffold comprises 42 or 49 to 56 sites available for binding a peptide.

According to some embodiments, the PEG molecule is a branched molecule, comprising at least two separate connections to a peptide. According to some embodiments, the PEG has 8 binding sites. According to other embodiments, the PEG is bound to additional PEG molecules. According to certain embodiments, multiple PEG molecules are bound to provide a multi-armed PEG molecule. According to some embodiments, eight 8-armed PEG molecules are bound to one central 8-armed PEG molecule to provide one PEG molecules with 56 sites capable of binding the peptides of the toxins of the present invention. According certain embodiments, the peptides are connected to the PEG scaffold through amide bonds formed between amino groups of an NH₂-PEG molecule. According to yet other embodiments, at least one peptide is connected to PEG scaffold though a Lys residue.

According to some embodiments, the peptides are bound to a PEG scaffold though a Lys residue.

According to some embodiments, the scaffold comprises a carbohydrate moiety.

According to some embodiments, the present invention provides a construct in which at least one of the peptides bound to PEG scaffold binds specifically to the antigen cMet and to at least one additional extracellular tumor antigens selected from EGFR, PD-L1, HER2, androgen receptor, benzodiazepine receptor, Cadherin, CXCR4, CTLA-4, CD2, CD19, endothelin receptor, ERBB4, FGFR, folate receptor, HER4, Mucin 1, OGFR, PD-1, PD-L2, PDGFR, and VEGFR. According to certain embodiments, at least one of the peptides bound to PEG scaffold binds specifically to cMet, and at least one peptide binds EGFR or PD-L1. According to some specific embodiments, the PEG scaffold comprises a peptide that binds cMet, a peptide that binds EGFR and a peptide that binds PD-L1.

According to one embodiment, the peptide that binds to cMet is a peptide having a sequence selected from the group consisting of: VGCVFVMSQKRC (M578, SEQ ID NO: 2), LSEGLGELMQRC (M582, SEQ ID NO: 1), CEPLLGEAWDLC (M571, SEQ ID NO: 3), CPRGLSGGWPAC (M572, SEQ ID NO: 4) and CESGEGTSNMDC (M573, SEQ ID NO: 5) or analog thereof. According to one embodiment, the peptide that binds to EGFR is a peptide having the sequence CHPGDKQEDPNCLQADK (SEQ ID NO: 11) or analog thereof. According to another embodiment, the peptide that binds specifically to PD-L1 is a peptide having CEGLPADWAAAC (SEQ ID NO: 12), or an analog thereof. According to certain embodiments, the construct comprises a peptide having the sequence LSEGLGELMQRC (SEQ ID NO: 1), a peptide having the sequence CHPGDKQEDPNCLQADK (SEQ ID NO: 11), and a peptide having the sequence CEGLPADWAAAC (SEQ ID NO: 12), wherein all peptides are bound to the scaffold.

According to some embodiments, the construct comprises at least one cyclic peptide. According to some embodiments, the targeting peptides bound to the scaffold directly or through a linker are cyclic.

According to some embodiments, the present invention provides a construct, wherein multiple copies of at least one of the peptides are connected to the scaffold. According to some embodiments, the scaffold comprises multiple copies of each of the at least two of the peptides. According to certain embodiments, the scaffold comprises multiple copies of a peptide that binds specifically to cMet, and multiple copies of a peptides that binds specifically to EGFR and/or PD-L1.

According to other embodiments, the toxin is bound to a carrier. The carrier may be as described herein above. Thus, according to one embodiment, the carrier is a scaffold. According to certain embodiments, the carrier is a peptidic scaffold.

According to other embodiments, the scaffold is PEG scaffold, i.e. formed from PEG. According to certain embodiments, the scaffold comprises a branched PEG molecule. According to some embodiments, the branched molecule comprises at least one available site to bind a toxin.

According to other embodiments, the scaffold comprises from 2 to 100, 3 to 90, 4 to 60, 5 to 50, 6 to 40, 7 to 35, 8 to 30, 9 to 25 or 10 to 20, or 2 to 50 sites that bind targeting peptides and/or a toxins. According to one embodiment, the construct comprises from 7 to 56, from 14 to 48, from 21 to 42 from 28 to 35, from 7 to 21 sites that bind a targeting peptide and/or a toxin. According to certain embodiment, the scaffold comprises 8 or 56, or 42 or 49 to 56 sites that bind a targeting peptide and/or a toxin.

According to some embodiments, the present invention provides a construct comprising a PEG scaffold connected to multiple copies of at least one toxin. According to some embodiments, the present invention provides a construct, comprising a scaffold connected to multiple copies of at least two toxins. According to some embodiments, the toxin is selected from the groups consisting of a toxin having the sequence CSARWGPTMPWC (SEQ ID NO: 15), a toxin having the sequence CRRGSRASGAHC (SEQ ID NO: 16), a toxin having the sequence MRPEIWIAQELRRIGDEFNE (BIMe toxin, SEQ ID NO: 13), a toxin having the sequence MRPEIWIAQELRRIGDEFNA (BIMa toxin, SEQ ID NO: 14), a toxin having the sequence SARWGPIMPW (SEQ ID NO: 17), a toxin having the sequence CSARWGPIMPWC (SEQ ID NO: 18), Diphtheria toxin, Pseudomonas exotoxin, Anthrax toxin, botulinum toxin, Ricin, PAP, Saporin, Gelonin, Momordin, ProTx-I ProTx-II, Conus californicus toxin, snake-venom toxin, cyanotoxin, and any combination thereof. According to some embodiments, the toxin is BIMe toxin (SEQ ID NO: 13).

According to any one of the above embodiments, the peptides and/or the toxin(s) are connected directly or through a spacer. According to other embodiments, the peptides and/or the toxin(s) are connected to the carrier, e.g. to a scaffold, through a spacer. According to some specific embodiments, the spacer comprises at least one amino acid residue.

According to any one of the above embodiments, the construct further comprises a permeability-enhancing moiety. The permeability-enhancing moiety may be connected directly to a peptide and/or to a toxin, or may be bound to the scaffold, optionally via a spacer. The term “permeability-enhancing moiety” refers to any moiety known in the art to facilitate actively or passively or enhance permeability of the compound through body barriers or into the cells. Non-limitative examples of permeability-enhancing moiety include: hydrophobic moieties such as fatty acids, steroids and bulky aromatic or aliphatic compounds; moieties which may have cell-membrane receptors or carriers, such as steroids, vitamins and sugars, natural and non-natural amino acids and transporter peptides, nanoparticles and liposomes. The term “permeability” refers to the ability of an agent or substance to penetrate, pervade, or diffuse through a barrier, membrane, or a skin layer.

According to another aspect, the present invention provides a composition comprising a construct of the present invention. According to one embodiment, the composition is a pharmaceutical composition. Thus, in some embodiments, the present invention provides a pharmaceutical composition comprising a construct of the present invention and a pharmaceutically acceptable excipient. All definitions, terms and embodiments of previous aspects are explicitly encompassed by this aspect.

The term “pharmaceutical composition” as used herein refers to a composition comprising the construct of the present invention as disclosed herein optionally formulated with one or more pharmaceutically acceptable excipients.

Formulation of the pharmaceutical composition may be adjusted according to applications. In particular, the pharmaceutical composition may be formulated using a method known in the art so as to provide rapid, continuous or delayed release of the active ingredient after administration to mammals. For example, the formulation may be any one selected from among plasters, granules, lotions, liniments, lemonades, aromatic waters, powders, syrups, ophthalmic ointments, liquids and solutions, aerosols, extracts, elixirs, ointments, fluidextracts, emulsions, suspensions, decoctions, infusions, ophthalmic solutions, tablets, suppositories, injections, spirits, capsules, creams, troches, tinctures, pastes, pills, and soft or hard gelatin capsules.

The term “pharmaceutically acceptable carrier” or “pharmaceutically acceptable excipient” as used herein refers to any and all solvents, dispersion media, preservatives, antioxidants, coatings, isotonic and absorption delaying agents, surfactants, fillers, disintegrants, binders, diluents, lubricants, glidants, pH adjusting agents, buffering agents, enhancers, wetting agents, solubilizing agents, surfactants, antioxidants the like, that are compatible with pharmaceutical administration. Non-limiting examples of suitable excipients are example, water, saline, phosphate buffered saline (PBS), dextrose, glycerol, ethanol, or the like and combinations thereof. Other suitable carriers are well known to those skilled in the art. The use of such media and agents for pharmaceutically active substances is well known in the art. The compositions may contain other active compounds providing supplemental, additional, or enhanced therapeutic functions.

The constructs of the present invention could be, according to some embodiments, suspended in a sterile saline solution for therapeutic uses. Numerous suitable drug delivery systems are known and include, e.g., implantable drug release systems, hydrogels, hydroxymethylcellulose, microcapsules, liposomes, microemulsions, microspheres, and the like. Controlled release preparations can be prepared through the use of polymers to complex or adsorb the molecule according to the present invention. For example, biocompatible polymers include matrices of poly(ethylene-co-vinyl acetate) and matrices of a polyanhydride copolymer of a stearic acid dimer and sebaric acid. The rate of release of the molecule according to the present invention from such a matrix depends upon the molecular weight of the molecule, the amount of the molecule within the matrix, and the size of dispersed particles.

The pharmaceutical composition of the present invention may be administered by any know method. The terms “administering” or “administration of” a substance, a compound or an agent to a subject can be carried out using one of a variety of methods known to those skilled in the art. For example, a compound or an agent can be administered, intravenously, arterially, intradermally, intramuscularly, intraperitonealy, intravenously, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods. In some embodiments, the administration includes both direct administration, including self-administration, and indirect administration, including the act of prescribing a drug. For example, as used herein, a physician who instructs a patient to self-administer a drug, or to have the drug administered by another and/or who provides a patient with a prescription for a drug is administering the drug to the patient.

According to some embodiments, the pharmaceutical composition is administered by an invasive mode of administration such as intramuscularly, intravenously, intra-arterially, intraarticulary or parenterally.

It will be apparent to those of ordinary skill in the art that the therapeutically effective amount of the molecule according to the present invention will depend, inter alia upon the administration schedule, the unit dose of molecule administered, whether the molecule is administered in combination with other therapeutic agents, the immune status and health of the patient, the therapeutic activity of the molecule administered and the judgment of the treating physician. As used herein, a “therapeutically effective amount” refers to the amount of a molecule required to alleviate one or more symptoms associated with a disorder being treated over a period of time.

Although an appropriate dosage of a molecule of the invention varies depending on the administration route, type of molecule (polypeptide, polynucleotide, organic molecule etc.) age, body weight, sex, or conditions of the patient, it will be determined by the physician in the end. Various considerations in arriving at an effective amount are described, e.g., in Goodman and Gilman's: The Pharmacological Bases of Therapeutics, 8th ed., Pergamon Press, 1990; and Remington's Pharmaceutical Sciences, 17th ed., Mack Publishing Co., Easton, Pa., 1990.

In one particular embodiment, the pharmaceutical composition of the present invention comprises a construct comprising at least two different peptides binding to at least two different extracellular tumor antigens, and at least one toxin, wherein the peptides and the toxin are covalently bound directly or through a carrier. According to some embodiments, at least one of the peptides binds specifically the receptor cMet and at least one of the peptides binds to an extracellular tumor antigen selected from EGFR and PD-L1. According to another embodiment, the other one of the at least two peptides binds specifically to an extracellular tumor antigen selected from the group consisting of EGFR, PD-L1, HER2, androgen receptor, benzodiazepine receptor, Cadherin, CXCR4, CTLA-4, CD2, CD19, endothelin receptor, ERBB4, FGFR, folate receptor, HER4, Mucin 1, OGFR, PD-1, PD-L2, PDGFR, and VEGFR. According to certain embodiments, the construct comprises from 2 to 10 different peptides. According to some embodiments, at least one of the peptides binds specifically to EGFR, and at least one of the peptides binds specifically to PD-L1. 2. According to some embodiments, the pharmaceutical composition comprises a construct comprising multiple copies of one, two or of three of the peptides. According to some embodiments, the construct comprises from 7 to 56, from 14 to 48, from 21 to 42, from 28 to 35, or from 7 to 21 copies of the each one of the peptide having the sequence CHPGDKQEDPNCLQADK (SEQ ID NO: 11) and CEGLPADWAAAC (SEQ ID NO: 12). According to some embodiments, the toxin is selected from the group consisting of a toxin binding to a eukaryotic elongation factor 2, BIM toxin, Diphtheria toxin, Pseudomonas exotoxin, Anthrax toxin, botulinum toxin, Ricin, PAP, Saporin, Gelonin, Momordin, ProTx-I ProTx-II, Conus californicus toxin, snake-venom toxin, and cyanotoxin. According to some embodiments, the BIM toxin consists of the sequence MRPEIWIAQELRRIGDEFNE (BIMe, SEQ ID NO: 13). According to certain embodiments, the toxin binding to eukaryotic elongation factor 2 is a toxin having the amino acid sequence selected from CSARWGPTMPWC (SEQ ID NO: 15) or CRRGSRASGAHC SEQ ID NO: 16), or an analog thereof. According to some embodiments, the construct comprises 2 to 10 different toxins. According to some embodiments, the construct comprises from 7 to 56, from 14 to 48, from 21 to 42 from 28 to 35, or from 7 to 21 copies of one or of two toxins. According to some embodiments, the construct comprises from 7 to 56, from 14 to 48, from 21 to 42 from 28 to 35, from 7 to 21 copies of the each one of the toxins. According to some embodiments, at least one of the targeting peptides is a cyclic peptides. According to some embodiments, the toxin is a cyclic peptide.

According to any one of the above embodiments, the pharmaceutical composition comprises a plurality of the constructs according to the present invention and according to the above embodiments.

According to another embodiment, the present invention provides a pharmaceutical composition according to the present invention, for use in treating a cell proliferative disease or disorder. According to some embodiments, the cell proliferative disease or disorder is cancer. Thus, according to one embodiment, the pharmaceutical composition of the present invention is for use in treating cancer.

The terms “treating” of “treatment of” a condition or patient refers to taking steps to obtain beneficial or desired results, including clinical results. Beneficial or desired clinical results include, but are not limited to, or ameliorating abrogating, substantially inhibiting, slowing or reversing the progression of a disease, condition or disorder, substantially ameliorating or alleviating clinical or esthetical symptoms of a condition, substantially preventing the appearance of clinical or esthetical symptoms of a disease, condition, or disorder, and protecting from harmful or annoying symptoms. Treating further refers to accomplishing one or more of the following: (a) reducing the severity of the disorder; (b) limiting development of symptoms characteristic of the disorder(s) being treated; (c) limiting worsening of symptoms characteristic of the disorder(s) being treated; (d) limiting recurrence of the disorder(s) in patients that have previously had the disorder(s); and/or (e) limiting recurrence of symptoms in patients that were previously asymptomatic for the disorder(s).

According to some embodiments, treating cancer comprises preventing or treatment tumor metastasis. According to certain embodiments, the metastasis is decreased. According to other embodiments, the metastasis is prevented.

According to some embodiments, treating cancer comprises increasing the duration of survival of a subject having cancer, comprising administering to the subject in need thereof a composition comprising a construct defined above whereby the administration of the construct increases the duration of survival.

According to some embodiments, treating cancer comprises increasing the progression of free survival of a subject having cancer.

According to some embodiments, treating cancer comprises increasing the duration of response of a subject having cancer. According to other embodiments, treating cancer comprises preventing tumor recurrence.

The cancer amendable for treatment according to the present invention includes, but not limited to: carcinoma, lymphoma, blastoma, sarcoma, and leukemia or lymphoid malignancies. More particular examples of such cancers include squamous cell cancer, lung cancer (including small-cell lung cancer, non-small cell lung cancer, adenocarcinoma of the lung, and squamous carcinoma of the lung), cancer of the peritoneum, hepatocellular cancer, gastric or stomach cancer (including gastrointestinal cancer), pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney or renal cancer, liver cancer, prostate cancer, vulval cancer, thyroid cancer, hepatic carcinoma and various types of head and neck cancer, as well as B-cell lymphoma (including low grade/follicular non-Hodgkin's lymphoma (NHL); small lymphocytic (SL) NHL; intermediate grade/follicular NHL; intermediate grade diffuse NHL; high-grade immunoblastic NHL; high-grade lymphoblastic NHL; high-grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome.

According to some embodiments, the cancer is selected from the group consisting of breast cancer, colorectal cancer, rectal cancer, non-small cell lung cancer, non-Hodgkins lymphoma (NHL), renal cell cancer, prostate cancer, liver cancer, pancreatic cancer, soft-tissue sarcoma, Kaposi's sarcoma, carcinoid carcinoma, head and neck cancer, melanoma, ovarian cancer, mesothelioma, and multiple myeloma. The cancerous conditions amendable for treatment of the invention include metastatic cancers.

According to other embodiments, the cancer is a solid cancer.

The pharmaceutical composition according to the present invention may be administered as a stand-alone treatment or in combination with a treatment with any other agent. According to a specific embodiment, constructs according to the present invention are administered to a subject in need thereof as part of a treatment regimen in combination with at least one anti-cancerous agent. The pharmaceutical composition according to the present invention may be administered in combination with the anti-cancerous agent or separately.

According to some embodiments, the activity of the construct of the present invention is synergistic to that of the additional anti-cancerous agent.

The pharmaceutical composition according to the present invention may be administered together with an anti-neoplastic composition. According to a specific embodiment, the anti-neoplastic composition comprises at least one chemotherapeutic agent.

The term “anti-neoplastic composition” refers to a composition useful in treating cancer comprising at least one active therapeutic agent capable of inhibiting or preventing tumor growth or function or metastasis, and/or causing destruction of tumor cells. Therapeutic agents suitable in an anti-neoplastic composition for treating cancer include, but not limited to, chemotherapeutic agents, radioactive isotopes, toxins, cytokines such as interferons, and antagonistic agents targeting cytokines, cytokine receptors or antigens associated with tumor cells.

A “chemotherapeutic agent” is a chemical compound useful in the treatment of cancer. Examples of chemotherapeutic agents include alkylating agents such as thiotepa and CYTOXAN™ cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e. g., calicheamicin, especially calicheamicin gamma1I and calicheamicin omegaI1 (e.g., Agnew, Chem Intl. Ed. Engl. 33:183-186 (1994)); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, ADRIAMYCIN™ doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK™ polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL™ paclitaxel (Bristol-Myers Squibb Oncology, Princeton, N.J.), ABRAXANE™ Cremophor-free, albumin-engineered nanoparticle formulation of paclitaxel (American Pharmaceutical Partners, Schaumberg, Ill.), and TAXOTERE™ doxetaxel (Rhone-Poulenc Rorer, Antony, France); chloranbucil; GEMZAR™ gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; NAVELBINE™ vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids such as retinoic acid; capecitabine; and pharmaceutically acceptable salts, acids or derivatives of any of the above.

Also included in this definition are anti-hormonal agents that act to regulate or inhibit hormone action on tumors such as anti-estrogens and selective estrogen receptor modulators (SERMs), including, for example, tamoxifen (including NOLVADEX™ tamoxifen), raloxifene, droloxifene, 4-hydroxytamoxifen, trioxifene, keoxifene, LY117018, onapristone, and FARESTON toremifene; aromatase inhibitors that inhibit the enzyme aromatase, which regulates estrogen production in the adrenal glands, such as, for example, 4(5)-imidazoles, aminoglutethimide, MEGASE™ megestrol acetate, AROMASIN™ exemestane, formestanie, fadrozole, RIVISOR™ vorozole, FEMARA™ letrozole, and ARIMIDEX™ anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signaling pathways implicated in aberrant cell proliferation, such as, for example, PKC-alpha, Raf and H-Ras; ribozymes such as a VEGF expression inhibitor (e.g., ANGIOZYME™ ribozyme) and a HER2 expression inhibitor; vaccines such as gene therapy DNA-based vaccines, for example, ALLOVECTIN™ vaccine, LEUVECTIN™ vaccine, and VAXID™ vaccine; PROLEUKIN™ rIL-2; LURTOTECAN™ topoisomerase 1 inhibitor; ABARELIX™ rmRH; and pharmaceutically acceptable salts, acids or derivatives of any of the above.

According to another aspect, the present invention provides a method of treating cancer in a subject in need thereof comprising administering to said subject a pharmaceutical composition of the present invention. According to one embodiment, the present invention provides a method of treating cancer in a subject in need thereof comprising administering to said subject a therapeutically effective amount of a construct or a peptide of the present invention. According to some embodiments, the pharmaceutical composition is administered as part of a treatment regimen together with at least one anti-cancer agent. The term “therapeutically effective amount” is an amount of a drug, compound, construct etc. that, when administered to a subject will have the intended therapeutic effect. The full therapeutic effect does not necessarily occur by administration of one dose, and may occur only after administration of a series of doses.

The terms “induce cell death” and “promote cell death” are used herein interchangeably and mean that the of the present invention (i.e. the peptide, the analog or the fragment) can directly inducing cell death to cells, where cell death includes apoptosis and necrosis. The cell death may be caused due to interaction of the compound of the present invention with molecules molecule expressed on the cell surface or with molecules located within the cell such as molecule located in the cytosol, bound to the inner side of the cell membrane, located in the organelles or present on the membrane of the organelles, either inner or outer part of it.

The term “cell death” as used herein encompasses both destruction and damage or impairment of cells. The term “cell death” encompasses cell ablation.

The term “conjugate” refers to any substance formed from the joining together or connecting of two or more molecules. In particular, the term conjugate encompasses a compound formed from connection of two or more peptides of any one of the above embodiments or a compound comprising said peptide bound to another molecule. According to some embodiments, the peptide or analog of the present invention is conjugated with a carrier protein or moiety which improves the peptide's antigenicity, solubility, stability or permeability. A fusion protein comprising at least one peptide according to the invention is also within this scope.

According to another embodiment, the conjugate comprises at least one peptide comprising or consisting of sequence selected from the group consisting of: LLCGRMIGDMSC (E9f, SEQ ID NO: 6), CDRTDAPASANC (E404, SEQ ID NO: 7), CDDDNAARDENC (E478, SEQ ID NO: 8), CQGGRLDLFGRC (E494, SEQ ID NO: 9), LCGYGETLMVSC (E133f, SEQ ID NO: 10), CHPGDKQEDPNCLQADK (E13.3, SEQ ID NO: 11), VGCVFVMSQKRC (M578, SEQ ID NO: 2), LSEGLGELMQRC (M582, SEQ ID NO: 1), CEPLLGEAWDLC (M571, SEQ ID NO: 3), CPRGLSGGWPAC (M572, SEQ ID NO: 4), CESGEGTSNMDC (M573, SEQ ID NO: 5), or analog thereof and another molecule. According to some embodiment, the molecule is selected from an active agent, an extracellular tumor antigen targeting molecule, a carrier, a toxin, a permeability-enhancing moiety and an anti-cancer agent.

The extracellular tumor antigen targeting molecule, a carrier, a toxin, an anti-cancer agent as defined according to the present invention. The term “active agent” refers to an agent that has biological activity, pharmacologic effects and/or therapeutic utility.

According to another aspect, the present invention provides a peptide that binds cMet comprising the amino acids sequence selected from the group consisting of: VGCVFVMSQKRC (M578, SEQ ID NO: 2), LSEGLGELMQRC (M582, SEQ ID NO: 1), CEPLLGEAWDLC (M571, SEQ ID NO: 3), CPRGLSGGWPAC (M572, SEQ ID NO: 4) and CESGEGTSNMDC (M573, SEQ ID NO: 5) or an analog or a salt thereof.

According to another aspect, the present invention provides a peptide that binds EGFR comprising the amino acids sequence selected from the group consisting of: LLCGRMIGDMSC (E9f, SEQ ID NO: 6), CDRTDAPASANC (E404, SEQ ID NO: 7), CDDDNAARDENC (E478, SEQ ID NO: 8), CQGGRLDLFGRC (E494, SEQ ID NO: 9), LCGYGETLMVSC (E133f, SEQ ID NO: 10), or an analog or a salt thereof.

According to other embodiments, the invention provides a peptide toxin comprising the sequence MRPEIWIAQELRRIGDEFNE (BIMe, SEQ ID NO: 13) or an analog or salt thereof.

According some embodiments, the peptide consists of 9 to 30 amino acids. According to other embodiments, each peptide consists of 8 to 25 amino acids. According to yet other embodiments, each peptide consists of 7 to 20 amino acids. According to some embodiments, each peptide consists of 8, 9, 10, 11, 12, 13, 14, 15, 16, 17, 18, 19 or 20 amino acids. Each possibility represents a separate embodiment of the invention.

According to some embodiments, the peptide or analog thereof is a cancer cells targeting peptide. Thus, in one embodiment, the peptide is for use in cancer cell targeting.

According to some embodiments, the conjugate comprises at least one peptide comprising and another molecule. According to one embodiment, the molecule is selected from an active agent, an extracellular tumor antigen targeting molecule, a carrier, a permeability-enhancing moiety, a toxin, an anti-cancer agent and a combination thereof.

some embodiments, the present invention provides a pharmaceutical composition comprising the peptide of the present invention, or the conjugate of the present invention.

All definitions and embodiments of other aspects of the present invention related to said peptides and conjugates are encompassed by this aspect as well.

According to some embodiments, the pharmaceutical composition is for treating a cell proliferative disease or disorder. According to some embodiments, cell proliferative disease or disorder is cancer. According to one embodiment, the pharmaceutical composition comprises a peptide, a conjugate or a construct as defined in any one of the embodiments of the present invention. Thus, in certain embodiment, the present invention provides a pharmaceutical composition comprising a peptide or a construct defined above for use in treating cancer.

According to another aspect, the present invention provides a method of treating a proliferative disease or disorder in a subject in need thereof comprising administering a therapeutically effective amount of the peptides, conjugates or constructs defined above.

According to another aspect, the present invention provides an isolated polynucleotide comprising a sequence encoding a peptide, analog or conjugate as defined in any one of the embodiments of the present invention.

According to another aspect, the present invention provides a nucleic acid construct, comprising the polynucleotide according to any one of the above embodiments. According to one embodiment, the polynucleotide is operably linked to a promoter.

The term “nucleic acid construct”, as used herein, refers to an artificially constructed segment of nucleic acid. It can be an isolated or integrated in another nucleic acid molecule.

As used herein, the term “operably linked”, “operably encodes”, and “operably associated” are used herein interchangeably and refer to the functional linkage between a promoter and nucleic acid sequence, wherein the promoter initiates transcription of RNA corresponding to the DNA sequence.

The term “promoter” is a regulatory sequence that initiates transcription of a downstream nucleic acid. The term “promoter” refers to a DNA sequence within a larger DNA sequence defining a site to which RNA polymerase may bind and initiate transcription. A promoter may include optional distal enhancer or repressor elements. The promoter may be either homologous, i.e., occurring naturally to direct the expression of the desired nucleic acid, or heterologous, i.e., occurring naturally to direct the expression of a nucleic acid derived from a gene other than the desired nucleic acid. A promoter may be constitutive or inducible. A constitutive promoter is a promoter that is active under most environmental and developmental conditions. An inducible promoter is a promoter that is active under environmental or developmental regulation, e.g., upregulation in response to xylose availability.

According to another aspect, the present invention provides a vector comprising the polynucleotide sequence or the nucleic acid construct of the present invention.

The terms “vector” and “expression vector” are used herein interchangeably and refer to any non-viral vector such as plasmid, cosmid, artificial chromosome (bacterial or yeast), or viral vector such as virus, retrovirus, bacteriophage, or phage, binary vector in double or single stranded linear or circular form, or nucleic acid, sequence which is able to transform host cells and optionally capable of replicating in a host cell. The vector may contain an optional marker suitable for use in the identification of transformed cells, e.g., tetracycline resistance or ampicillin resistance. According to one embodiment, the vector is a plasmid. According to another embodiment, the vector is a phage or bacteriophage.

The term “plasmid” refers to circular, optionally double-stranded DNA capable of inserting a foreign DNA fragment to a cell and optionally capable of autonomous replication in a given cell. Plasmids usually contain further sequences in addition to the ones, which should be expressed, like marker genes for their specific selection and in some cases sequences for their episomal replication in a target cell. In certain embodiments, the plasmid is designed for amplification and expression in bacteria. Plasmids can be engineered by standard molecular biology techniques.

According to another aspect, the present invention provides a cell comprising the polynucleotide comprising a sequence encoding the peptide as defined in any one of the embodiments of the present invention.

The terms “comprising”, “comprise(s)” “include(s),” “having,” “has,” “contain(s),” as used in this specification means “consisting at least in part of”. When interpreting each statement in this specification that includes the term “comprising”, features other than that or those prefaced by the term may also be present. Related terms such as “comprise” and “comprises” are to be interpreted in the same manner. The terms “have”, “has”, having” and “comprising” may also encompass the meaning of “consisting” and “consisting essentially of”, and may be substituted by these terms. The term “consisting of” excludes any component, step or procedure not specifically delineated or listed. The term “consisting essentially of” means that the composition or component may include additional ingredients, but only if the additional ingredients do not materially alter the basic and novel characteristics of the claimed compositions or methods.

As used herein, the term “about”, when referring to a measurable value such as an amount, a temporal duration, and the like, is meant to encompass variations of +/−10%, or +/−5%, +/−1%, or even +/−0.1% from the specified value.

The following examples are intended to illustrate how to make and use the compounds and methods of this invention and are in no way to be construed as a limitation. Although the invention will now be described in conjunction with specific embodiments thereof, it is evident that many modifications and variations will be apparent to those skilled in the art. Accordingly, it is intended to embrace all such modifications and variations that fall within the spirit and broad scope of the appended claims.

### EXAMPLES

Reference is now made to the following examples, which together with the above descriptions, illustrate the invention in a non-limiting fashion.

### Example 1. Preparation of PEG Complex Loaded With a Targeting Peptide to cMet or EGFR and With a Toxin

Constructs of a branched PEG molecule covalently coupled with a single cancer-targeting moiety and one kind of toxin moiety were designed and synthesized. The targeting moieties included the cMet binding peptides M582 (SEQ ID NO: 1), M571 (SEQ ID NO: 3), M572 (SEQ ID NO: 4) and M573 (SEQ ID NO: 5), and the EGFR binding peptides E9f (SEQ ID NO: 6), E404 (SEQ ID NO: 7), E478 (SEQ ID NO: 8), E494 (SEQ ID NO: 8) and E133f (SEQ ID NO: 10). The toxin peptide was BIMe (SEQ ID NO: 13).

The preparation method comprised two steps. At the first step a branched PEG-NHS containing eight arms was produced in which seven arms were coupled with targeting or toxin protected peptides, and one with a Lysine residue protected with FMOC (fmoc-Lys). The synthesis ended with removal of the fmoc protecting group. At the second step, eight of the peptide/toxin-PEG molecules produced in step 1 were coupled to another branched PEG-NHS molecule of eight arms to obtain a construct of multi-branched PEG coupled with 56 toxin/targeting moieties, of which 49 moieties are connected with the toxin peptide BIMe (SEQ ID NO: 13) and 7 are a targeting peptide.

### Example 2. Preparation of Multi-Armed PEG Complex Loaded With Three Targeting Peptides and One Toxin

A construct of a branched PEG molecule covalently coupled with three different cancer-targeting peptides and one toxin peptide was designed and synthesized. The targeting peptides included in this example construct were the cMet binding peptide M582 (SEQ ID NO: 1), the EGFR binding cyclic peptide E13.3 (SEQ ID NO: 11), the PD-L1-binding cyclic peptide GR (SEQ ID NO: 12), and the toxin peptide was BIMe (SEQ ID NO: 13).

The preparation method comprised two steps. At the first step a branched PEG-NHS containing eight arms was produced in which seven arms were coupled with targeting/toxin moieties (E13.3 protected with dde) and one with a Lysine residue protected with FMOC (fmoc-Lys). The synthesis ended with removal of the fmoc protecting group. At the second step, eight of the peptide/toxin-PEG molecules produced in step 1 were coupled to another branched PEG-NHS molecule of eight arms to obtain a construct of multi-branched PEG coupled with 56 toxin/targeting moieties, of which 35 moieties are connected with the toxin peptide BIMe (SEQ ID NO: 13), and 21 are targeting peptides (7 copies of each of: cMet targeting peptide M582, EGFR targeting peptide E13.3 and PD-L1 targeting peptide PD-L1-GR). The final step involved removal of the dde protecting group from the sequence of the E13.3 peptide (SEQ ID NO: 11). The detailed synthesis protocol was as following:

### Step 1—Preparation of Branched PEG Coupled With One Type of Targeting or Toxin Moiety

2.4 μmole of a targeting peptide or 7.3 μmole of toxin peptide were dissolved in DMSO.

All peptides have only one primary amine, except for E13.3 (SEQ ID NO: 11), which has 3, of which one is protected with dde, and the N-terminal is blocked with an acetate residue. 5.9 mg Fmoc-Lys-OH (Novabiochem Cat. Num. 852023; MW=368.43) was dissolved in 150 μl of HCl 0.1 M, followed by addition of 650 μl of DMSO to reach a concentration of 20 mM.

33.4 mg of 8-arm star PEG-NHS (Mw 10 KDa, Creative Biotechnologies) were dissolved in 167 μl of dioxane to reach a concentration of 20 mM.

Each of the targeting peptides solutions were mixed with 17 μl of Fmoc-Lys-OH solution and 17 μl of PEG solution.

Each of the toxin peptides solutions were mixed with 52 μl of Fmoc-Lys-OH solution and 52 μl of PEG solution. Each mix was supplemented with TEA (trimethylamine) to 5%. Each solution was incubated for 15.5 hours at room temperature on a Rotamix at 30 rpm to obtain a clear solution of 8 armed PEG coupled with 7 molecules of a specific targeting/toxin moiety and one arm containing a primary amine (The fmoc protection is removed in this process to give one free primary amine on each PEG molecule).

The branched PEG-peptide molecules are denoted PEG-M582, PEG-E13.3, PEG-PD-L1-GR, PEG-Tox1 and PEG-Tox2.

### Step 2—Construction of Multi-Branched PEG Construct Coupled to 56 Targeting/Toxin Moieties

The branched PEG-peptide solutions: PEG-M582, PEG-E13.3, PEG-PD-L1-GR, PEG-BIMe were mixed together with 20 mM PEG-NHS solution in a stoichiometric molar ratio of PEG-NHS:PEG-M582:PEG-E13.3:PEG-PD-L1-GR:PEG-BIMe of 1:1:1:1:5 and incubated for 2 hours at room temperature on a Rotamix at 30 rpm, followed by slow addition of 80% hydrazine to a final concentration of 5%. Hydrazine was used to remove the dde protecting group from the E13.3 sequence (SEQ ID NO: 11). The mixture was incubated for 2 hours at room temperature on a Rotamix at 30 rpm. The resultant construct is a multi-branched PEG coupled with 56 targeting/toxin moieties: 7 copies of M582 peptide (SEQ ID NO: 1), 7 copies of E13.3 peptide (SEQ ID NO: 11), 7 copies of PD-L1-GR peptide (SEQ ID NO: 12), 35 copies of BIMe (SEQ ID NO: 13). At the end of the reaction, PBS was added with gentle mixing.

### Step 3—Ultrafiltration

The samples were ultrafiltrated with two additions of 20 ml PBS using Vivaspin 20 concentrator (30 K MWCO PES) to a concentration of ˜206 μM of loaded multi-armed PEG denoted as PEG-M582-E13.3-(PD-L1-GR)-BIMe, and the buffer was substituted to PBS.

In a similar way, additional multi-branched PEGs carrying alternative toxins or targeting peptides were produces.

### Example 3. Toxicity of Constructs Comprising EGFR-Binding Peptides

The constructs prepared in Examples 1 and 2 have been used.

### Cells Culture and Seeding

A431 cells (human squamous carcinoma cells express about 100,000 copies of EGFR on each cell) and MCF-7 cells (breast cancer cell expressing about 3,000 copies of EGFR on each cell) are thawed and cultivated to achieve exponentially growing cultures. Cells are collected, counted and seeded at the density of 7,000 cells/well and 5,000 cells/well, respectively, in a 96 well tissue culture plate.

The plates were incubated until the next day at the following conditions: 37±1° C., humidified, and 5±0.5% CO₂/air, to enable cells adherence to the wells.

### Treatment

The cell viability was tested using Alamar Blue viability assay. At the next day following the seeding, growth media was replaced with 200 μl assay media that contained 2% FBS and test items at different concentrations of the construct (1, 3 and 8 μM), or vehicle control (PBS; concentration-0). Plates were incubated at 37±1° C., humidified 5±0.5% CO₂/air. After 48 hours of incubation, images of the cells of the different treatments are taken on microscope.

### Example 4. Effect of Constructs Comprising cMet-Binding Peptides on the Growth and Viability of A549 Cells

### Materials and Methods

The constructs PEG-M582-BIMe, PEG-M571-BIMe, PEG-M572-BIMe and PEG-M573-BIMe, prepared as described in Example 1. The constructs comprise PEG having 56 arms: 7 arms carrying a targeting peptide to cMet, and 49 arms carrying the toxin BIMe (SEQ ID NO: 13). PBS was used as a negative control and the construct PEG-E404-GW2 (carrying the peptides of SEQ ID NO: 7 and SEQ ID NO: 18) was used as a positive control.

A-549 cells (human lung tumor cells, overexpressing the extracellular receptors cMet, EGFR and PD-L1), were thawed and cultivated to achieve exponentially growing cultures. Cells were collected, counted and seeded in a 96 well tissue culture plate at the following densities: A-549: 5,000 cells/well. The plate was incubated until the next day at 37±1° C., humidified, 5±0.5% CO₂/air, to enable cells adherence to the wells.

### Treatment and Results

At the next day after the seeding, growth media were replaced with solutions of tested constructs in assay medium (growth media containing 2% FBS). Solutions containing the tested constructs were applied carefully (onto the sides of the well, not directly onto the cells) in volume of 200 μl/well to achieve the final concentrations of 1, 3 or 9 μM. The plate was incubated at 37±1° C., humidified 5±0.5% CO₂/air.

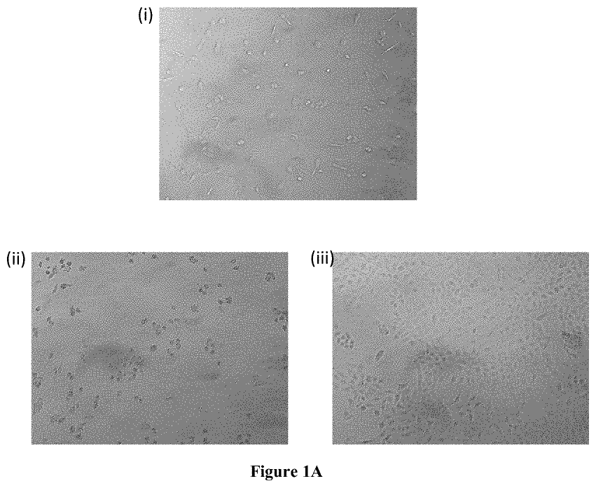

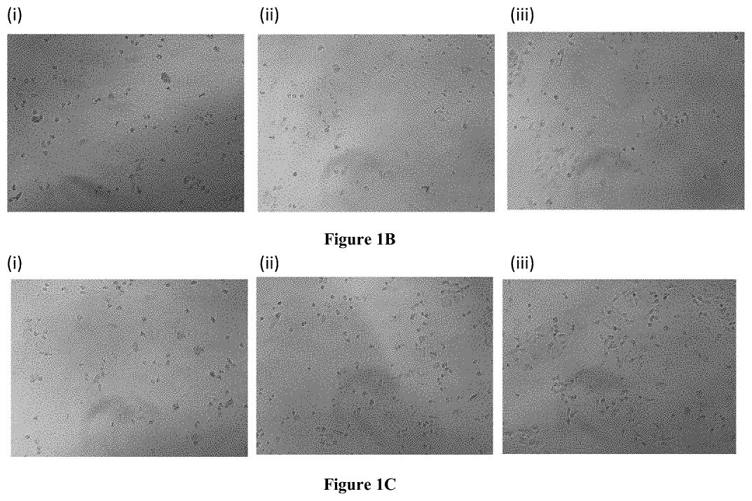

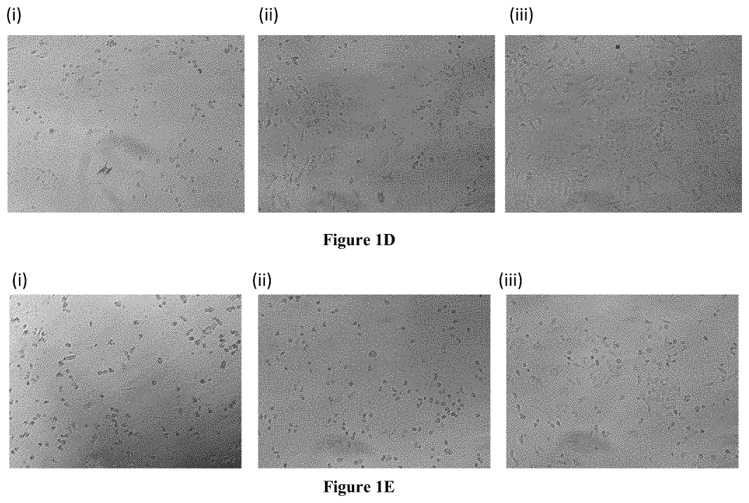

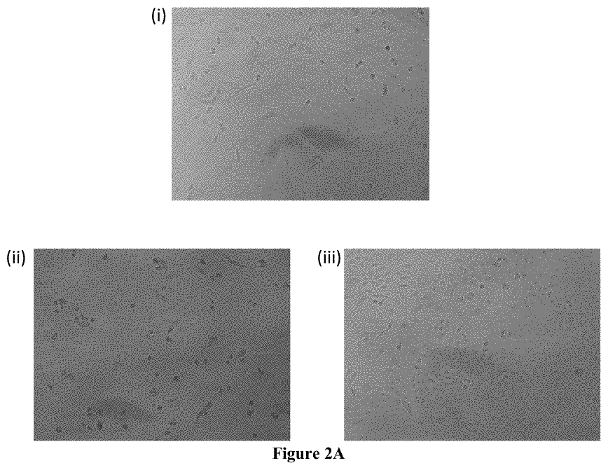

After 48 hours of incubation, images of cells from tested and control groups were taken by microscope. Representative images are presented on FIGS. 1A-1E. As can be seen in the images, the constructs comprising cMet-targeted peptides were effective in inducing cell death in all concentrations tested.

### Example 5. Effect of Constructs Comprising EGFR-Binding Peptides on the Growth and Viability of A549 Cells

### Materials and Methods

The test items, constructs PEG-E9f-BIMe, PEG-E404-BIMe, PEG-E478-BIMe, PEG-E494-BIMe and PEG-E133f-BIMe were prepared as described in Example 1. The constructs comprise PEG having 56 arms: 7 arms carrying a targeting peptide to EGFR and 49 arms carrying the toxin BIMe (SEQ ID NO: 13). PBS was used as a negative control and the construc PEG-E404-GW2 (carrying the peptides of SEQ ID NO: 7 and SEQ ID NO: 18) as a positive control.

A549 cells (human lung tumor cells, overexpressing the receptors cMet, EGFR and PD-L1), were thawed and cultivated to achieve exponentially growing cultures. The cells were collected, counted and seeded in a 96 well tissue culture plate at 5,000 cells/well. The plate was incubated until the next day at 37±1° C., humidified, 5±0.5% CO₂/air, to enable cells adherence to the wells.

### Treatment and Results

At the next day after seeding, growth media were replaced with test item solutions prepared in assay medium (2% FBS). Test item solutions were applied carefully (onto the sides of the well, not directly onto the cells) in a volume of 200 μl/well to achieve the final concentrations of 1, 3 or 9 μM. The plate was incubated at 37±1° C., humidified 5±0.5% CO₂/air.

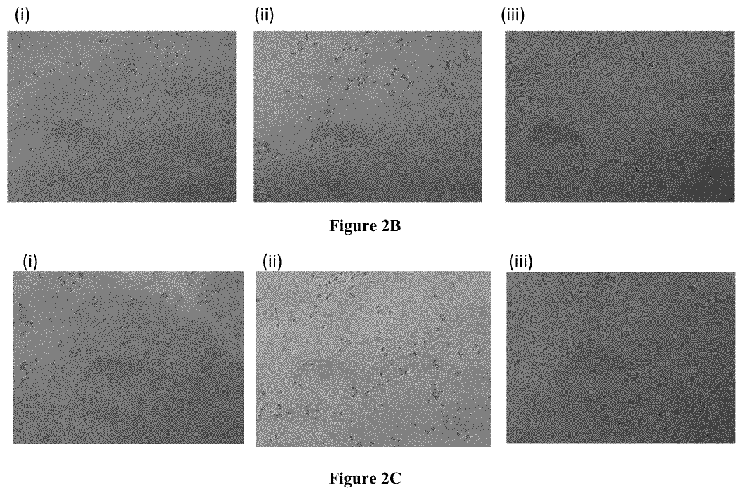

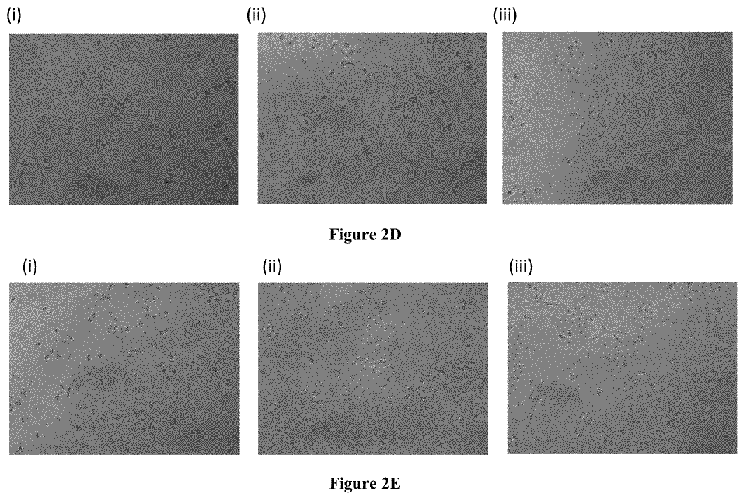

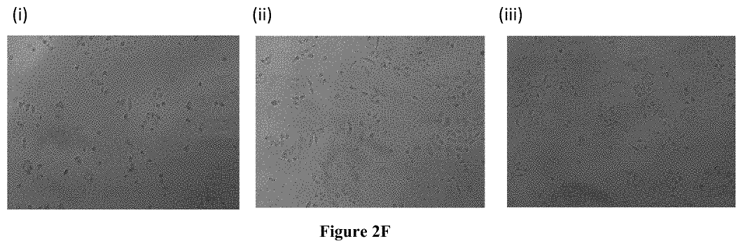

After 48 hours of incubation, images were taken on microscope. The results of representative images are shown in FIGS. 2A-2F. As can be seen in the images, the constructs comprising EGFR-targeted peptides were effective in inducing cell death in all concentrations tested.

### Example 6. Effect of Constructs Comprising Combination of EGFR-Targeted, PD-L1-Targeted and cMet-Targeted Peptides on the Growth and Viability of A549 Cells

### Materials and Methods

The construct PEG-E13.3-GR-M582-BIMe was prepared as described in Example 2. The construct comprises 56-armed PEG carrying 7 copies of each of the targeting peptides E13.3 (SEQ ID NO: 11), GR (SEQ ID NO: 12) and M582 (SEQ ID NO: 1), and 35 copies of the toxin BIMe (SEQ ID NO: 13). PBS was used as a negative control and PEG-E404-GW2 (carrying the peptides of SEQ ID NO: 7 and SEQ ID NO: 18) as a positive control.

A-549 cells (human lung tumor cells, overexpressing the receptors cMet, EGFR and PD-L1) were thawed and cultivated to achieve exponentially growing cultures. Cells were collected, counted and seeded in a 96 well tissue culture plate at the following densities: A-549: 5,000 cells/well. The plate was incubated until the next day at 37±1° C., humidified, 5±0.5% CO₂/air, to enable cells adherence to the wells.

### Treatment

At the next day after seeding, growth media were replaced with solutions containing the tested constructs or controls in assay medium (2% FBS). Solution was applied carefully (onto the sides of the well, not directly onto the cells) in volume of 200 μl/well to achieve the final concentrations of 0.1, 0.3, 1 or 3 μM. The plate was incubated at 37±1° C., humidified 5±0.5% CO₂/air.

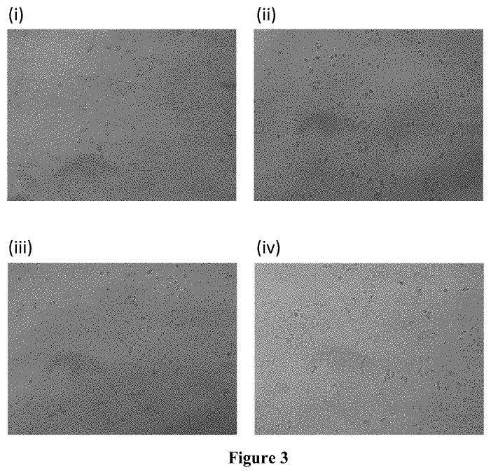

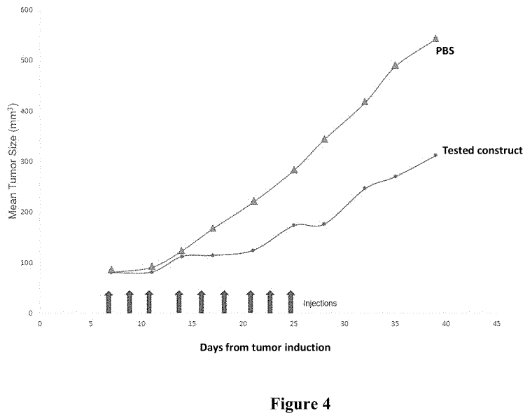

After 48 hours of incubation, images of treatment and control groups were taken on microscope. The results of representative images are shown in FIG. 3.

### Results

As can be seen from the image of FIG. 3, compared to the control presented in FIG. 2A, PEG-E13.3-GR-M582-BIMe was effective in killing A549 cells in concentrations of 3, 1, 0.3 and even 0.1 μM. The number of cells after treatment with 0.1 μM of PEG-E13.3-GR-M582-BIMe was less than a half as compared to the negative control, indicating that the IC50 is lower than 0.1 μM.

### Example 7. Evaluation of Antitumor Effect of the Constructs In Vivo

### Material and Methods

Animals: 18 athymic female nude mice 6-7 weeks old are divided into 3 groups (1 control group and 2 test items groups) are allowed to accumulate for at least 5 days. Following accumulation, A431 tumor cells are subcutaneously injected to right flan region of each mouse, the day of injection is denoted Day 0.

The following parameters are monitored: weight (twice a week) and tumor size (width and length are measured with digital caliper and the tumor volume is calculated as width²×length/2).

When the tumor reaches the size of 100-150 mm3, mice are subjected to 3 IV injections of test items during the first week. Animals are observed from additional 3 weeks. Following observation period, mice are euthanized, the tumor is excised, measured and fixed in 4% formaldehyde solution for further analysis.

### Example 8. Stability of the Peptides in Bovine Serum

The stability of the selected peptides in bovine serum is assessed by measuring the inhibitory activity of the peptides (at 50 ng/ml) after incubation of the peptides with bovine serum at 37° C. for different periods of time.

### Example 9. Stability of the Peptides in Mice

The stability of the fluorescently marked peptides alone or bound to PEG is evaluated in vivo by injecting the compounds to the tail vein of mice. The blood of the animals is analyzed at different time intervals, for the presence of the fluorescent peptide.

### Example 10. Accumulation of Fluorescently-Labeled Peptide-PEG Complexes in Cancerous Tumors

Peptide-PEG constructs are labeled with Fluorescein and injected IV to Xenograft mice bearing subcutaneous NCI-H1650 tumor (lung cancer model). Following anesthesia, kidney, liver and tumor are collected at specific time points and the fluorescence is measured.

### Example 11. Xenograft Mice In Vivo Model

Female Hsd:Athymic Nude-Foxn1nu mice, 6-7 weeks of age at tumor induction were obtained from accredited breeder. A suspension of A549 tumor cells (human epithelial lung carcinoma that overexpresses EGFR, PD-L1 and cMet receptors, ATCC, CCL-185), was injected to n=12 animals at dose volume of 0.2 ml/animal (5×10⁶ cells/animal) by a single subcutaneous (SC) injection into the right flank area, midway between the axillary and inguinal regions.

The tested compound was a 56-arm PEG construct that contained 7 copies of a EGFR-targeting peptide (E13.3 SEQ ID NO: 11), 7 copies of a PD-L1 targeting peptide (GR, SEQ ID NO: 12), 7 copies of a cMet targeting peptide (M582, SEQ ID NO: 1), and 35 copies of the toxic peptide BIMe (SEQ ID NO: 13).

The tested construct and the control (PBS) were injected to n=6 tumor-bearing mice (initial tumor size of ˜80 mm³ and up) per group by the intravenous (IV) route, at dose volume of 4 ml/kg. Test materials were injected 9 times (on weekdays only) during a period of three weeks (every 2-3 days). The dose of the tested construct was 36 mg/kg, equivalent to the killing concentration in the in vitro experiment of the same batch (data not shown).

Monitoring of progressive changes in tumor growth were carried out in all animals twice a week from measurable tumors until study termination, using Electronic Digital Calipers. The tumor volume was determined and calculated according to the following equation: V (mm3)=d2 (mm2) ^{a} D (mm)/2. The symbols d and D represent the smallest and the largest perpendicular tumor diameters, respectively.

Viability checks, for mice mortality and morbidity, were performed at least once daily. Cage-side observation for the detection of abnormalities were also performed once daily. Whenever an abnormality was detected it was recorded. Determination of individual body weights of animals were made shortly before tumor induction (Day 0) and twice weekly thereafter.

FIG. 9 describes the results of the mice xenografts experiment. The results show that the tested construct inhibited the growth of the cancer tumors by 42% at the end of the experiment (P<0.05 by T-test). No health deterioration was observed with any of the mice.

Although the present invention has been described herein above by way of preferred embodiments thereof, it can be modified, without departing from the spirit and nature of the subject invention as defined in the appended claims.

1. A construct comprising multiple copies of at least one peptide that binds specifically to the extracellular tumor antigen cMet, multiple copies of two other peptides binding to at least two different extracellular tumor antigens, and at least one toxin, wherein the peptides consists of from 8 to 30 amino acid residues, and wherein the peptides and the toxin are covalently bound directly or through a carrier or a scaffold.

2. The construct of claim 1, wherein at least one of the other peptides binds specifically to an extracellular tumor antigen selected from human epidermal growth factor receptor (EGFR) and human Programmed death-ligand 1 (PD-L1).

3. The construct of claim 2, wherein one of the other peptides binds specifically to EGFR and the other one binds specifically to PD-L1.

4. The construct of claim 1, wherein the peptide that binds specifically to cMet is selected from the group consisting of: SEQ ID Nos. 1-5.

5. The construct of claim 2, wherein the peptide that binds specifically to EGFR is selected from the group consisting of: SEQ ID Nos. 6-11.

6. The construct of claim 2, wherein the peptide that binds specifically to EGFR is a peptide having the sequence CHPGDKQEDPNCLQADK (SEQ ID NO: 11) or an analog thereof, and the peptide that binds specifically to PD-L1 is a peptide having the sequence CEGLPADWAAAC (SEQ ID NO: 12) or an analog thereof

7. The construct of claim 1, wherein the toxin is a peptide, polypeptide or protein toxin selected from the group consisting of: a toxin specifically binding to a eukaryotic elongation factor 2, Diphtheria toxin, Pseudomonas exotoxin, Anthrax toxin, botulinum toxin, Ricin, PAP, Saporin, Gelonin, Momordin, ProTx-I ProTx-II, Conus californicus toxin, snake-venom toxin, and cyanotoxin.

8. The construct of claim 7, wherein the toxin binding to eukaryotic elongation factor 2 is selected from a peptide of SEQ ID NO: 13 and SEQ ID NO. 14.

9. The construct of claim 1, wherein the construct comprises from 3 to 10 different peptides, from 2 to 10 different toxins and 2 to 50 copies of each of the peptides and the toxins.

10. The construct of claims 1, wherein the peptide that bind specifically cMet is set forth in SEQ ID NO: 1, the peptide that binds specifically to EGFR is is set forth in SEQ ID NO: 11, the peptide binds specifically to PD-L1 is set forth in SEQ ID NO: 12 and the toxin is a peptide set forth in SEQ ID NO: 13.

11. The construct of claim 10 comprising 7 copies of each of the targeting peptides and 35 copies of the toxin.

12. The construct of claims 1, wherein at least one of the peptides and/or at least one toxin is covalently bound to an organic scaffold directly or through a linker or spacer.

13. The construct of claim 12, wherein the organic scaffold comprises a polyethylene glycol (PEG) molecule or a modified PEG molecule.

14. The construct of claim 13, wherein the PEG molecule comprises 8 to 56 sites available to bind the peptides and the toxin(s).

15. A pharmaceutical composition comprising a construct according to claim 1, and a pharmaceutically acceptable excipient.

16. A method of treating cancer in a subject in need thereof comprising administering to said subject a pharmaceutical composition according to claim 15.

17. A peptide comprising an amino acid sequence selected from the group consisting of:
(SEQ ID NO: 1)
LSEGLGELMQRC;
(SEQ ID NO: 2)
VGCVFVMSQKRC;
(SEQ ID NO: 3)
CEPLLGEAWDLC;
(SEQ ID NO: 4)
CPRGLSGGWPAC;
and
(SEQ ID NO: 5)
CESGEGTSNMDC;
(SEQ ID NO: 6)
LLCGRMIGDMSC;
(SEQ ID NO: 7)
CDRTDAPASANC;
(SEQ ID NO: 8)
CDDDNAARDENC;
(SEQ ID NO: 9)
CQGGRLDLFGRC;
and
(SEQ ID NO: 10)
LCGYGETLMVSC;
((SEQ ID NO: 13)
MRPEIWIAQELRRIGDEFNE;
or an analog, conjugate or salt thereof.

18. A pharmaceutical composition comprising the peptide of claim 17 or a conjugate thereof, and a pharmaceutically acceptable carrier.

19. A method of treating cancer in a subject in need thereof comprising administering a therapeutically effective amount of the peptide, analog, conjugate or salt of claim 17.

20. An isolated polynucleotide comprising a sequence encoding the peptide or analog thereof according claim 17, or a construct or vector comprising said polynucleotide.
